(19) United States
(12) Patent Application Publication
(10) Pub. No.: US 2025/0129417 A1
(43) Pub. Date: Apr. 24, 2025

(54) ULTRAFAST MOLECULAR INVERSION PROBE-BASED TARGETED SEQUENCING ASSAY FOR LOW VARIANT ALLELE FREQUENCY

(71) Applicant: YEDA RESEARCH AND DEVELOPMENT CO. LTD., Rehovot (IL)

(72) Inventors: Liran SHLUSH, Herzliya (IL); Tamir BIEZUNER, Rehovot (IL)

(21) Appl. No.: 18/683,896

(22) PCT Filed: Aug. 18, 2022
(86) PCT No.: PCT/IL2022/050907

§ 371 Date: Feb. 15, 2024

Related U.S. Application Data
(60) Provisional application No. 63/234,523, filed on Aug. 18, 2021.

(51) Int. Cl. C12Q 1/6874

(52) U.S. Cl. CPC C12Q 1/6874

(57) ABSTRACT

Provided herein is an improved molecular inversion probe protocol, exhibiting reduced noise, high specificity and sensitivity and improved coverage at GC-rich regions.

### FIELD OF THE INVENTION

Provided herein is an improved molecular inversion probe protocol, exhibiting reduced noise, high specificity and sensitivity and improved coverage at GC-rich regions.

### BACKGROUND ART

[1] Chastain E. C. Kulkarni S., Pfeifer J. Clinical Genomics. 2015; Boston: Academic Press; 37-55.
[2] Boyle E. A., et al., MIPgen: optimized modeling and design of molecular inversion probes for targeted resequencing. Bioinformatics. 2014; 30:2670-2672.
[3] Hiatt J. B., et al., Single molecule molecular inversion probes for targeted, high-accuracy detection of low-frequency variation. Genome Res. 2013; 23:843-854.
[4] Almomani R., et al., Evaluation of molecular inversion probe versus Truseq® custom methods for targeted next-generation sequencing. PLOS One. 2020; 15: e0238467.
[5] Park G., et al., Characterization of background noise in capture-based targeted sequencing data. Genome Biol. 2017; 18:136.
[6] Ma X., et al., Analysis of error profiles in deep next-generation sequencing data. Genome Biol. 2019; 20:50.
[7] Acuna-Hidalgo R., et al., Ultra-sensitive sequencing identifies high prevalence of clonal hematopoiesis-associated mutations throughout adult life. Am. J. Hum. Genet. 2017; 101:50-64.

Acknowledgement of the above references herein is not to be inferred as meaning that these are in any way relevant to the patentability of the presently disclosed subject matter.

### BACKGROUND OF THE INVENTION

The development of next-generation sequencing (NGS) approaches has revolutionized molecular biology research as they can generate large volumes of sequencing data per run, however it has yet to be wildly implemented into clinical practice. While complete omics approaches (whole genome/transcriptome/epigenome) provide opportunity for novel discoveries, they are still not cost-effective and therefore are not routinely used as diagnostic tools. To democratize NGS to a large number of samples and applications in a cost- and time-effective manner, several targeted enrichment approaches have been developed. Furthermore, deep sequencing aimed at identifying low variant allele frequency (VAF) mutations is usually based on targeted sequencing approaches.

With the growing demand for high performance and cost-effective targeted sequencing technologies it is generally required to choose between scalability (both for number of samples and number of both targets) and cost. Currently there are no targeted sequencing approaches that are both scalable, cost effective, simple and fast. Hybrid capture has high-performance but is still costly and time consuming [1]. On the other hand, amplicon sequencing is simple and cost effective but is not scalable for large number of targets.

Molecular Inversion Probe (MIP) technology enables targeting multiple genomic regions and generating a sequencing library in economical, one pot reaction [2, 3]. Although MIP technology can potentially be fully automated and scalable, its main downsides are its low performance (i.e. uniformity [1], [3], reduced coverage at GC-rich regions [4]). Another drawback of the MIP technology is the lack of an accurate noise model, an essential tool for low VAF analysis.

The library preparation step of any targeted sequencing approach has a unique issue of background error signatures which correlate with the specific chemistry and various steps of the protocol [5]. It is therefore required to comprehensively understand the intrinsic background noise of the technology and to generate a noise model to determine if suspected variants are real [6]. The state-of-the-art in MIP low VAF analysis is the algorithm published by Acuna-Hidalgo et al. [7]. While this study introduces a new statistical approach to call low VAF variants based on a Poisson noise model it has several caveats such as the lack of extensive validation and the use of technical duplicates which were separated at the final step of the MIP protocol while true technical duplicates were not used. These drawbacks leave background noise model of the MIP protocol without a cross platform validation, and uncertainty regarding its accuracy.

There is an unmet need for a simple and fast targeted sequencing protocol, and specifically an improved molecular inversion probe protocol, which exhibits shortened turn-around-time from DNA sample to an NGS library, high precision and sensitivity at low VAF and improved GC reach regions coverage.

### SUMMARY OF THE INVENTION

Disclosed herein is a molecular inversion probe (MIP)-based targeted sequencing method which is advantageous over MIP-based targeted sequencing known to date.

The method disclosed herein address the main drawbacks of MIP, as detailed below. By analyzing and modeling the MIP protocol noise in depth; and modifying the current MIP biochemistry to enhance poor performance and noise properties the MIP protocol steps were recalibrated, and an improved MIP protocol was designed. Advantageously this protocol, also termed hereinafter “iMIP”, was reduced to under three and a half hours (end to end). As a result, the iMIP protocol demonstrated a significantly lower background error rate compared to the known MIP protocols.

Moreover, using the iMIP protocol significantly reduces the number of false positive variants. Additional benefits rendered by the iMIP protocol include: less small families (<5) and more large families (>5), a significant increase in the median MIPs that work in the iMIP protocol compared to MIP protocol (609 versus 558 respectively p<0.00001; FIG. 2B); a significant improvement of in panel uniformity (FIG. 2C) and the on-target rate (FIG. 2D); significantly higher variant allele frequency (VAF) correlation between duplicates (FIG. 7B); significantly higher coverage across GC rich regions; and significantly higher uniformity (FIG. 3B).

As exemplified herein, the identified variants were subjected to amplicon sequencing using MIP designed for this purpose. Surprisingly, Amplicon sequencing yielded significantly reduced error rate in all possible single nucleotide variants (SNV) alterations.

Furthermore, applying a machine learning variant caller trained on the MIP dataset used in the iMIP protocol disclosed herein, resulted with significant improvement in precision from 16.67% (p=0.004) to 56.25% (p=1.4E-5) in correctly calling variant allele frequency, namely, VAF>0.005, compared to state of the art (Acuna-Hidalgo et al., Am J Hum Genet, 101, 50-64, 2017).

A first aspect of the present disclosure relates to a molecular inversion probe-based targeted sequencing method, specifically, an improved method. In some embodiments the disclosed method comprises the following steps:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

A further aspect of the present disclosure relates to a method for diagnosing a pathological disorder in a subject by identifying at least one genetic and/or epigenetic variation/s and/or at least one nucleic acid sequence of at least one pathogenic entity associated with the pathologic disorder in at least one target nucleic acid sequence of at least one sample of the subject. More specifically, the method comprising the step of performing molecular inversion probe-based targeted sequencing in at least one test sample of the subject or in any nucleic acid molecule obtained therefrom. It should be understood that the presence of one or more of the variation/s in at least one target nucleic acid sequence and/or the presence of at least one nucleic acid sequence of the pathogenic entity, indicates that the subject has a risk, is a carrier, or is suffering from the pathologic disorder. In some embodiments, the molecular inversion probe-based targeted sequencing method performed herein comprises the following steps. One step (a) involves contacting at least one molecular inversion probe (MIP) with at least one target nucleic acid sequence of the subject that may contain the genetic variation associated with the disorder, or the at least one nucleic acid sequence of the pathogenic entity and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

A further aspect of the present disclosure relates to a method of detecting the presence of one or more target microorganism or an infectious entity, for example, pathogenic entity in a test sample. More specifically, the method comprising the step of performing molecular inversion probe-based targeted sequencing in at least one nucleic acid molecule obtained from the sample. It should be noted that the presence of one or more target nucleic acid sequence associated with the microorganism or infectious entity in the sample indicates the presence thereof in the sample. In some embodiments, the molecular inversion probe-based targeted sequencing method applicable in the disclosed detection methods, comprising the step of:

One step (a) involves contacting at least one nucleic acid molecule of the sample with at least one MIP specific for at least one target nucleic acid sequence associated with the microorganism or pathogenic entity and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction buffer for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

A further aspect of the present disclosure relates to a method of determining the genotype and/or the genetic profile of at least one nucleic acid molecule of at least one organism and/or at least one infectious entity, for example, at one or more loci of interest. More specifically, the method comprising the step of performing molecular inversion probe-based targeted sequencing in at least one test sample comprising the at least one nucleic acid molecule. More specifically, the molecular inversion probe-based targeted sequencing method used herein comprising the step of:

In one step (a), contacting at least one MIP with at least one target nucleic acid sequence, e.g., a target sequence comprising one or more loci of interest, and incubating for a hybridization time of one to three and a half hours. In more specific embodiments, the MIP used in the disclosed methods may comprise: (i) a first region comprising a first sequence complementary to a first target region in the target nucleic acid sequence, and (ii) a second region comprising a second sequence complementary to a second target region in the target nucleic acid sequence. The first hybridization step results in MIP/s hybridized to the first and second target regions of the target nucleic acid sequence, that comprises the one or more polymorphic loci of interest.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

A further aspect of the present disclosure relates to a method for identifying low variant allele frequency (VAF) mutations in a target nucleic acid molecule by performing molecular inversion probe-based targeted sequencing in said nucleic acid molecule. More specifically, the method comprising the step of:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the polymerization and/or ligation reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

A further aspect of the present disclosure relates to a method for performing molecular inversion probe-based targeted sequencing in at least one target nucleic acid sequence comprising at least one GC-rich region, the method comprising the step of:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the polymerization and/or ligation reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1A-1B. Increased background error rate in the MIP protocol results in high false positive rate which can be improved by machine learning algorithms

FIG. 1A. presents distribution per base background error rate (log 10) of each possible alteration comparing the molecular inversion probe (MIP) protocol (dark, left side) and Amplicon sequencing protocol (light, right side). Mann-Whitney-Wilcoxon test two-sided with Bonferroni correction ns: 5.00e-02<p<=1.00e+00, *: 1.00e-02<p<=5.00e-02, **: 1.00e-03<p<=1.00e-02, ***: 1.00e-04<p<=1.00e-03, ****: p<=1.00e-04.

FIG. 1B. presents performance (sensitivity, precision and specificity) calculated for the state-of the art Poisson distribution error suppression method (“Poisson”, left columns, black) and for a machine learning variant caller trained on the inventor's entire MIP dataset (“MIP”, right columns, white). Variants from the MIP protocol were validated by amplicon sequencing and true positives were defined based on the results of the amplicon sequencing. The precision of the machine learning variant caller (MIP) to detect variants with variant allele frequency (VAF)>0.005 was significantly better, Fischer exact test p=1.4E-5.

FIG. 2A-2D. An improved MIP (iMIP) protocol has reduced background error rate and improved sequencing quality attributes

FIG. 2A. presents background error rate calculated for iMIP (gray) MIP (dark gray) and amplicon (light gray).

FIG. 2B. presents the number of MIP targets that worked across the selected samples between MIP and iMIP, Mann-Whitney-Wilcoxon test two-sided with Bonferroni correction P_val<10\hat{ }−217 (****).

FIG. 2C. presents uniformity of MIP and iMIP across the selected samples, Mann-Whitney-Wilcoxon test two-sided with Bonferroni correction P_val<10\hat{ }−11 (****).

FIG. 2D. presents on target rate across the selected samples, Mann-Whitney-Wilcoxon test two-sided with Bonferroni correction P_val<10\hat{ }−131 (****).

FIG. 3A-3C. The iMIP protocol has better coverage and uniformity across GC-rich regions

FIG. 3A. presents MIP (n=535 samples) vs iMIP (n=905 samples) comparison of GC-rich genes coverage (GC-rich targets have higher than 55% GC content). Targets which are part of each gene were included, the data is normalized by: sum (targets depth)/number of targets/original FASTQ reads *100. Other than SETBP1 all p values were significant (****: P≤0.001). Mann-Whitney-Wilcoxon test two-sided with Bonferroni correction.

Note: the values are in log scale, and for visualization, zero values were omitted.

FIG. 3B. presents uniformity between MIP and iMIP across GC-rich targets P_val<10\hat{ }−15 (****). Mann-Whitney-Wilcoxon test two-sided with Bonferroni correction.

FIG. 3C. presents the coverage of the MIP and iMIP protocols across CEBPA, depth was normalized as in FIG. 3A. Note: the values are in log scale, and for visualization, zero values were omitted.

FIG. 4A-4F. The iMIP protocol can successfully capture a genotyping panel of 8349 targets

FIG. 4A. presents the use of iMIP protocol to sequence 170 samples across 8349 targets, where median on target rate was 95% and was correlated with the number of reads in FASTQ.

FIG. 4B. presents comparison (Mann-Whitney-Wilcoxon test) between the uniformity of the genotyping panel and of the ARCH panel.

FIG. 4C. presents the median depth across all targets of the genotyping panel.

FIG. 4D. presents the number of targets in the genotyping panel that have a certain copy number of ligation and extension arms (as calculated by MIPgen software): MIPs were divided into groups: 1:1-ligation and extension arms have one copy in the genome, 1 in 1 (1 in one)—one of the arms (either ligation or extension) has one copy in the genome, 1<and<100-both ligation and extension arms have between 1 to 100 copies, >100-both arms have above 100 copies. Left bar-percentage of MIPs in each of the groups, out of the total panel. Right bar-percentage of the read across all data.

FIG. 4E. presents median depth compared between the target groups based on arms copy number.

FIG. 4F. presents performance of improved genotyping panel (reduced probes with high copy number in ligation and extension arms): Boxplots were calculated for 104 samples and the values that are presented are % targets with depth of at least one read, 10 reads, 50 reads and 100 reads.

FIG. 5. ROC curve of the Support vector machine (SVM) model

The figure presents the sensitivity of low VAF support vector machine (SVM) detection model as a ROC curve. Samples that were validated and had a p_value of zero were considered true. Train and test set were T=31 F=77113 for the train set and T=11 F=41528 for the test set.

FIG. 6A-6B. Samples from MIP and iMIP had similar distribution of original fastq reads count

FIGS. 6A and 6B. present MIP (6A) and iMIP (6B) protocol performance in samples that had similar depth FASTQ files, respectively (4-10M reads). reads depth distribution was evaluated per protocol. Distributions were similar based on two different statistical assays: Kolmogorov-Smirnov for 2 samples, two-sided p_value=0.2157 and Epps-Singleton p_value=0.2550.

FIG. 7A-7B. iMIP has higher correlation between VAF and VAFdup in duplicated mutations and larger UMI families

FIG. 7A. presents family size distribution in MIP and iMIP protocol. Family size at each MIP was calculated per unique molecular identifier (UMI) across MIP and iMIP samples. X axis defines the family size, and the Y axis defines how many families were identified for each family size 1-4 and greater than 5. Differences between MIP and iMIP were tested by the Mann-Whitney-Wilcoxon test: P_val<=1.00e-04.

FIG. 7B. The correlation between VAF and VAFdup (minimum 0.005, and minimum depth 100 for both duplicates) was calculated for all positions in which duplicates were identified for both iMIP and MIP samples.

Fitting a linear function between samples of MIP and iMIP between resulted in significant higher correlation between duplicates of the iMIP protocol MIP y=0.8524*x+0.0431 with R2=0.6849 iMIP y=0.8517*x+0.0528 with R2=0.7134 (Fisher's z, z=4.9595, p-value=0.0000).

FIG. 8A-8C. MIPs targeting GC-content below 55% have better overall performance

FIG. 8A. presents MIPs that provided poor coverage (Mean read depth <50 across all samples) in the MIP protocol (upper panel) and their corresponding performance in the iMIP protocol (lower panel). MIPs are sorted based on GC content.

FIGS. 8B and 8C. present uniformity and mean depth, respectively, in GC low and rich regions (bellow and above 55% GC content, respectively). Mann-Whitney-Wilcoxon test: p value=6.541e-69 and p value=2.577e-68, respectively for 8B and 8C.

FIG. 9. Key differences between iMIP and previous MIP protocols.

FIG. 10. iMip protocol has a reduced background error rate regardless of batch effects

Graph showing the background error rate for each alteration and each of the different batches (runs) of the MIP and iMIP protocols. iMIP batch is the right-hand side of each alternation column, as can be seen in the T->A example. The bimodal error rate of c->A seen in all batches of the MIP protocol, disappeared in the iMIP protocol. The left-side of each alternation column represents a batch that had a different MIP protocol than the standard MIP protocol and ran in a Nextseq 500 Instrument while all other batches ran in Novaseq 6000 Instrument, including the iMIP batch.

FIG. 11A-11B. Background error rate in single base indels. Graph showing background error rate in single base indels of mutations acquired from Varscan (FIG. 11A) and mutations from platypus (FIG. 11B) for MIP and iMIP protocol.

FIG. 12A-12C. Similar or improved uniformity and on target rates modified hybridization protocols

FIG. 12A and FIG. 12B Present a comparison of uniformity and on-target rates (respectively) for a range of dNTPs concentration for both 153 minutes (iMIP protocol) and iMIP modified protocol wherein the hybridization time is 103 minutes. A range of dNTPs concentration was examined for both hybridization protocols (for reference, the standard dNTPs concentration in the iMIP protocol matches 0.059 mM in the plots). Panel used is a cancer panel in a size of 31 probes. Each duplicate was averaged, all averaged sample had between 50K-120K total reads.

FIG. 12C. Comparison of normalized uniformity and on-target rates in iMIP modified protocol wherein the hybridization time was 135 minutes compared to 103 minutes. Panels used are either SNP or ARCH: ISP146 designate a SNP panel in a size of 161 probes; ISP170 designate a subset of ARCH panel in a size of 339 probes; ISP173 designate a complete ARCH panel in a size of 773 probes; SP178 designate a SNP panel in a size of 248 probes; The data of each sample was normalized by dividing the sample on-target % and uniformity % by the mean of the 103 minutes replicates of each experiment per panel. The 135 minutes program was found to have a significantly higher on-target % using the Mann Whitney U test (p-Value=0.016) while the uniformity did not show significant improvement.

FIG. 13. Similar uniformity and on target rates in shorter exonuclease inactivation period

Comparison of uniformity and on-target rates in iMIP modified protocol wherein the exonuclease inactivation conditions were for 5 minutes at 80° C., 90° C. or 95° C. As indicated above, the inactivation of the exonuclease in the iMIP protocol was 20 minutes at 80° C. The panel for this analysis is an ARCH panel in a size of 597 probes.

### DETAILED DESCRIPTION OF THE INVENTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

In the description and claims of the application, the words “include” and “have”, and forms thereof, are not limited to members in a list with which the words may be associated.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Disclosed herein is a two directional (i.e. statistical and biochemical) approach for the improvement of the MIP technology, a previously low performance but highly scalable and economical technology. To achieve this goal, the noise pattern of the technology was studied in large dataset and created a benchmark amplicon-based sequencing strategy to validate the candidate variants. This further improved the state-of-the-art algorithm for MIP noise reduction and generated a high precision low VAF machine learning calling model. The noise was further reduced by changing the protocol timing and enzymes.

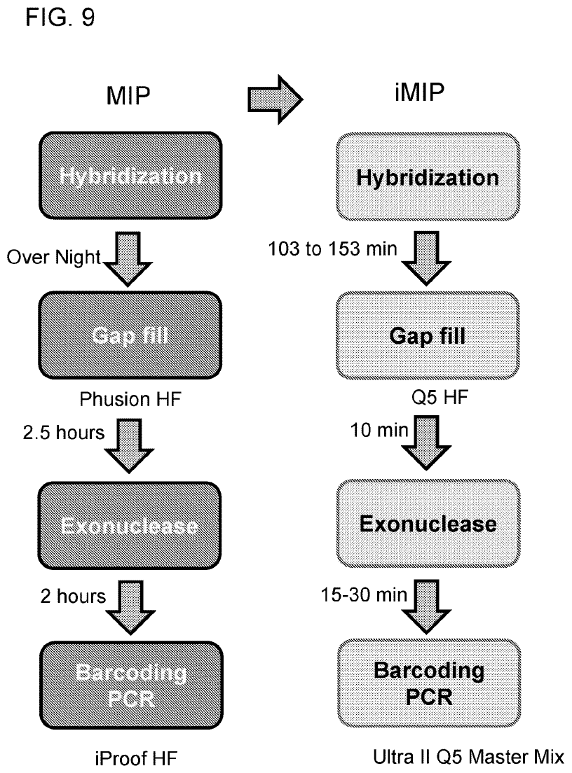

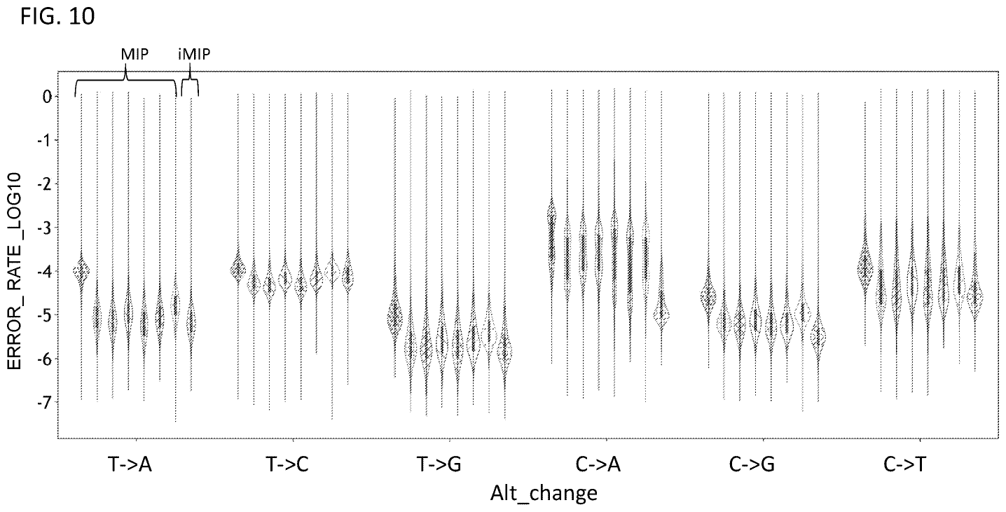

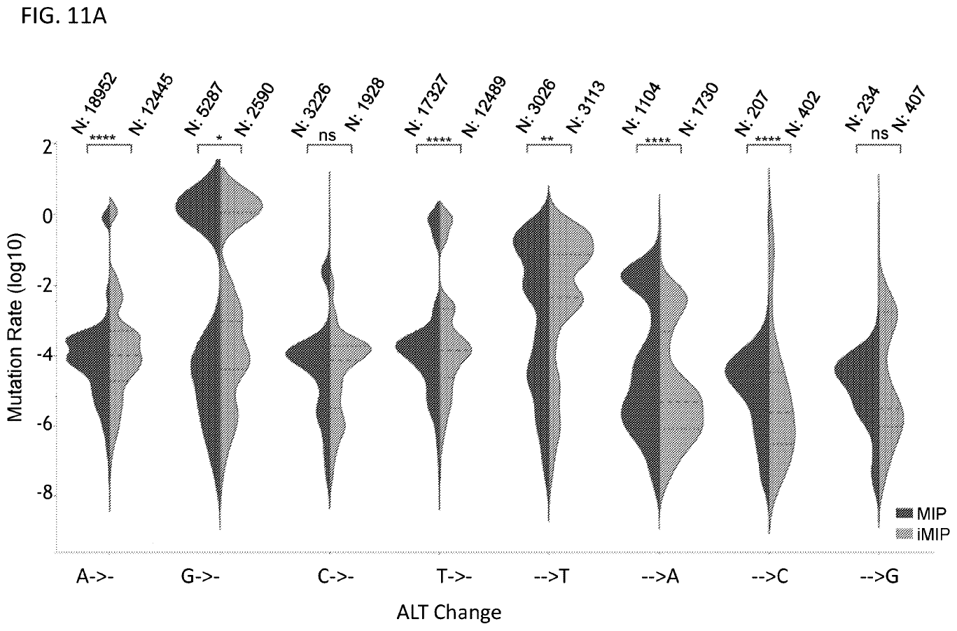

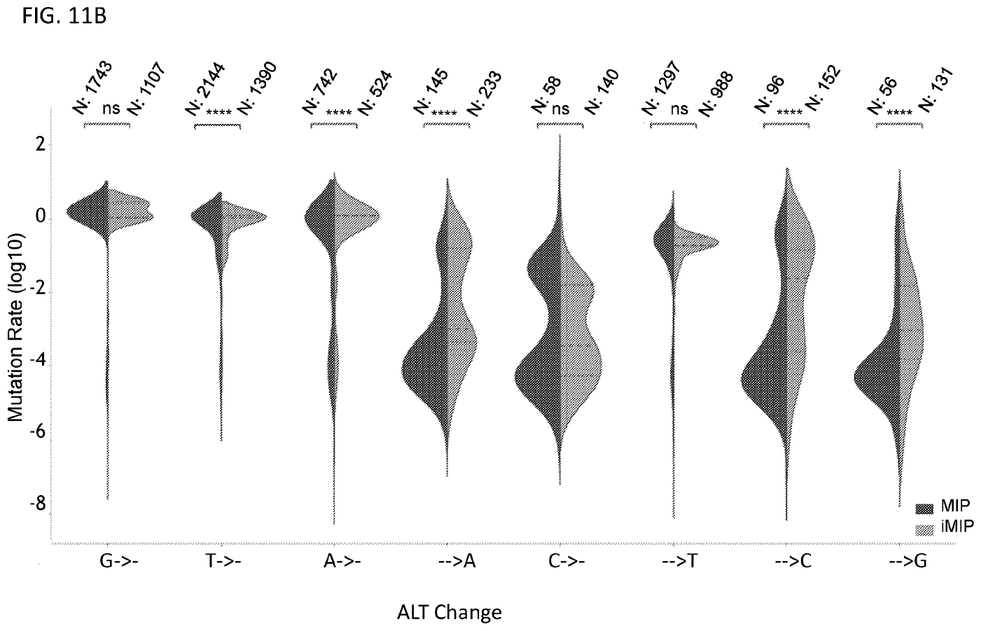

FIG. 9 summarizes the main differences between the improved MIP protocol of the present disclosure (iMIP), and previous MIP protocols. In brief, and as further detailed herein, the main advantages of the iMIP protocol are: (1) Shorter hybridization incubation of about 2.5 hours or less (instead of overnight); (2) Gap filing using Q5 High-Fidelity (HF) DNA Polymerase which takes approximately 10 minutes (instead of 2.5 hours); (3) Enzymatic digestion of linear probes and any other linear nucleic acid sequences present in the reaction mixture, is performed by adding Exonuclease I and Exonuclease III followed by 15 to 30 minutes incubation (instead of 2 hours); and (4) Amplification of final product, for example, using Ultra II Q5 Master Mix. Background error rate was calculated for each alteration and for each of the different batches (NGS runs) of the MIP and iMIP protocols). As demonstrated in FIG. 10, the iMIP protocol has a reduced background error rate regardless of batch. The bimodal error rate of C->A seen in all batches of the MIP protocol, disappeared in the iMIP protocol. Background error rate in single base indels of mutations acquired from Varscan and from platypus are presented in FIGS. 11A and 11B panels, respectively.

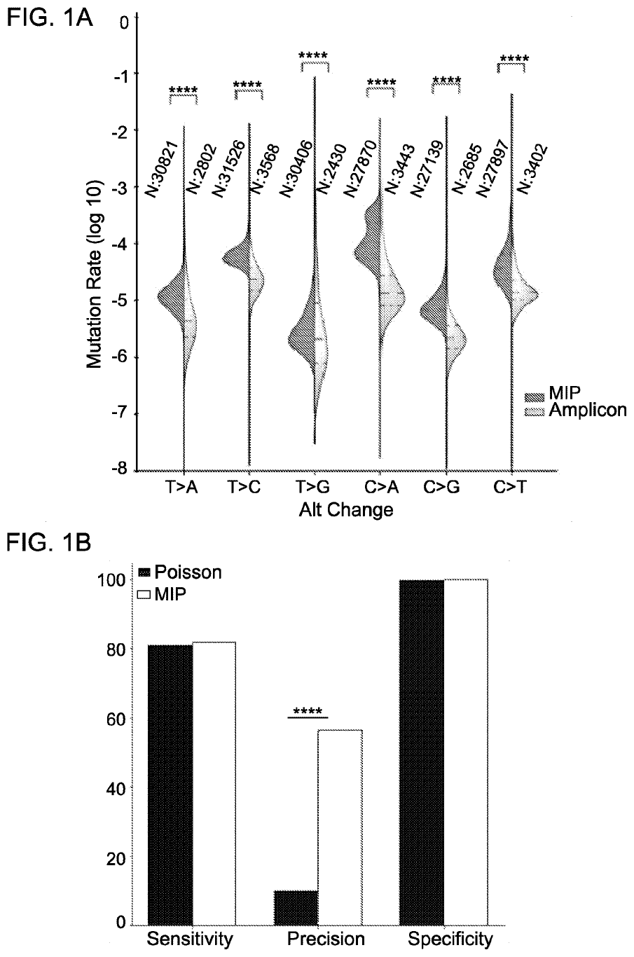

The improved iMIP protocol aided in the reduction of overall SNV noise of all possible alternations and eliminated the bimodal noise of C>A alternations (see, for example, FIG. 1A). Without being bound by any theory or mechanism of action. Thus, the short iMIP protocol of less than three and a half hours, is attractive for both clinical laboratories and large-scale screening efforts.

Calling low VAF using the MIP protocol could be further improved by utilizing unique molecular identifiers (UMI)/molecular tags (Waalkes, A., et al., (2017) Haematologica, 102, 1549-1557).

Although the disclosed MIP structure is composed of UMIs (7 nucleotides), the inventors chose not to use it. This is mainly because UMI utilization for low VAF requires higher depth per target that allows large number of families with size >5 (Shugay, M., (2017), PLoS Comput. Biol., 13, e1005480).

The inventors chose to allocate each sample ˜2 million reads and accordingly the vast majority of the families had a size <5. Nevertheless, it was also shown in the past that using a correct statistical model in hybrid capture protocols, enables correct VAF calling without the need for UMI correction (Abelson S., et al., Nature. 2018; 559:400-404), and the inventors have provided similar evidence here for the MIP protocol. The inventor's model is therefore suitable for detection of variants with VAF as low as 0.5% with sensitivity of 80% and significantly higher precision. If lower VAFs or higher sensitivity are needed, deeper sequencing will be used with the addition of UMI collapsing. However, in many instances this is not needed, and the disclosed protocol can answer the need for a cost effective low VAF protocol. The disclosed model and protocol can be generalized for every MIP panel and can be combined with UMI error correction, however for much deeper sequencing (which might be needed for minimal residual disease detection) the number nucleotides in the UMI should be increased correlatively to depth and VAF thresholds. While deep targeted sequencing has its own needs in the early diagnosis of cancer and other applications, the vast majority of targeted sequencing applications do not require low VAF detection and still suffer from high costs, long and complicated protocols. The disclosure of the subject invention provides a three and a half-hour single tube fully automated protocol which is now ready for clinical use as its performance is significantly improved.

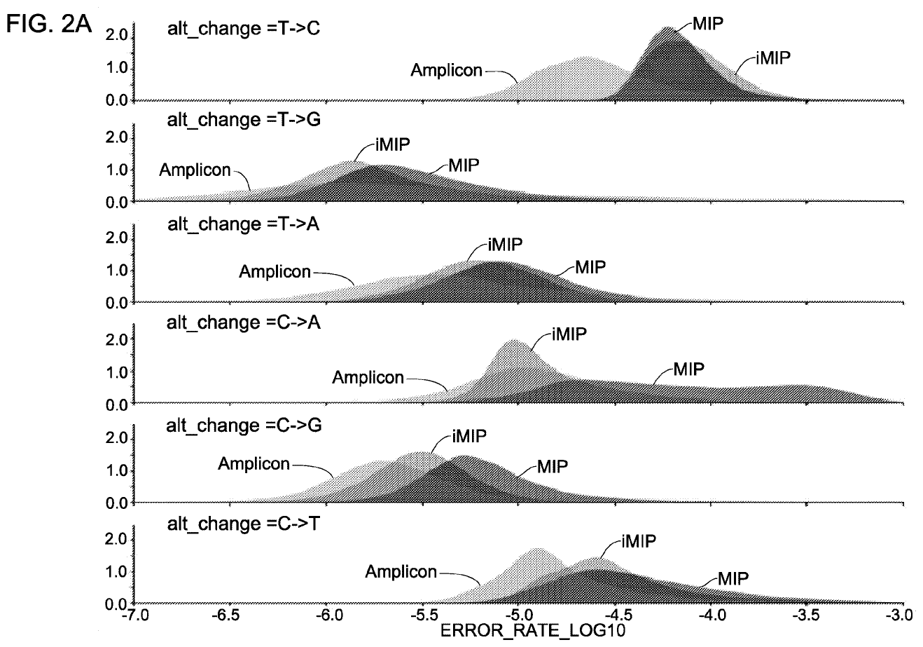

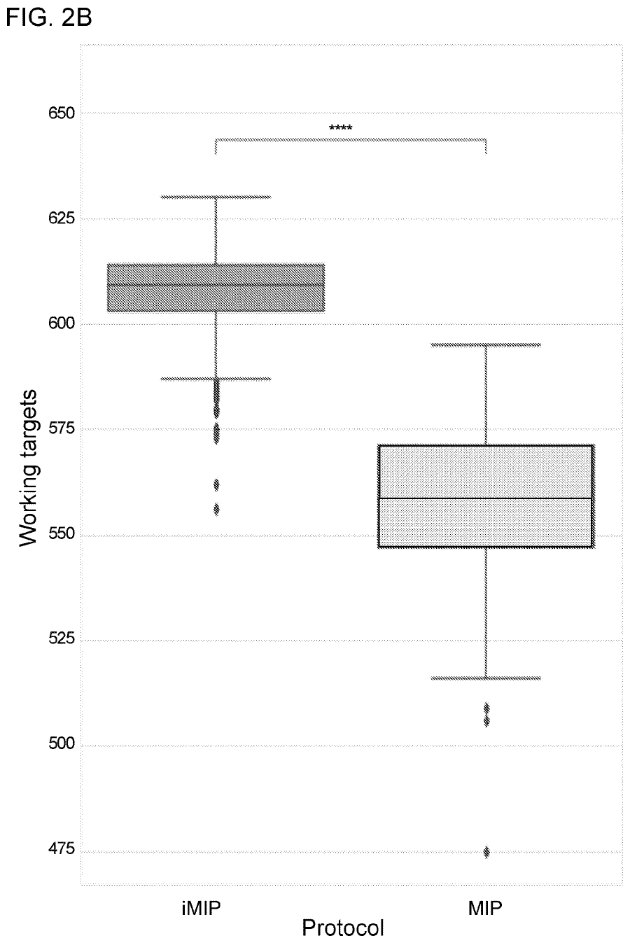

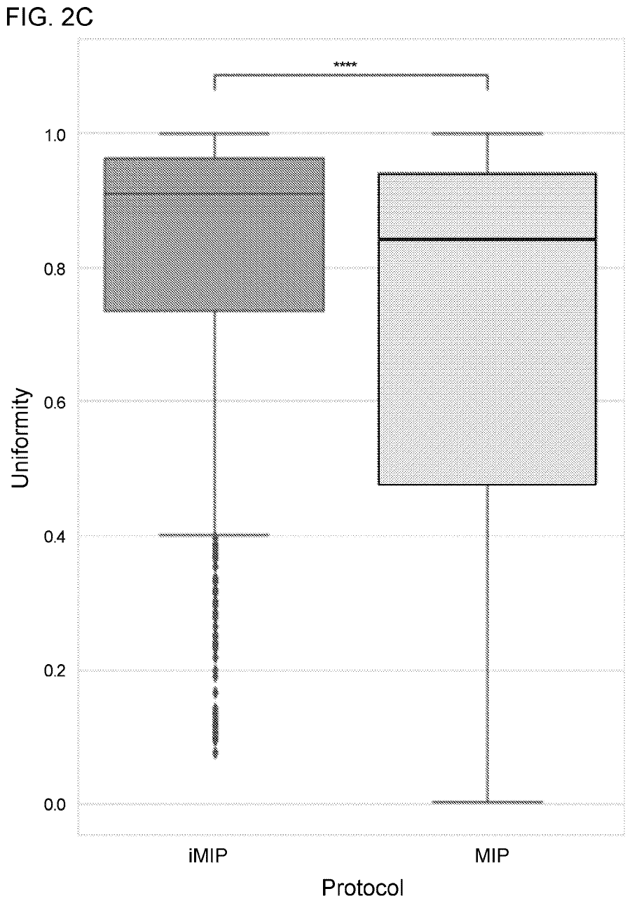

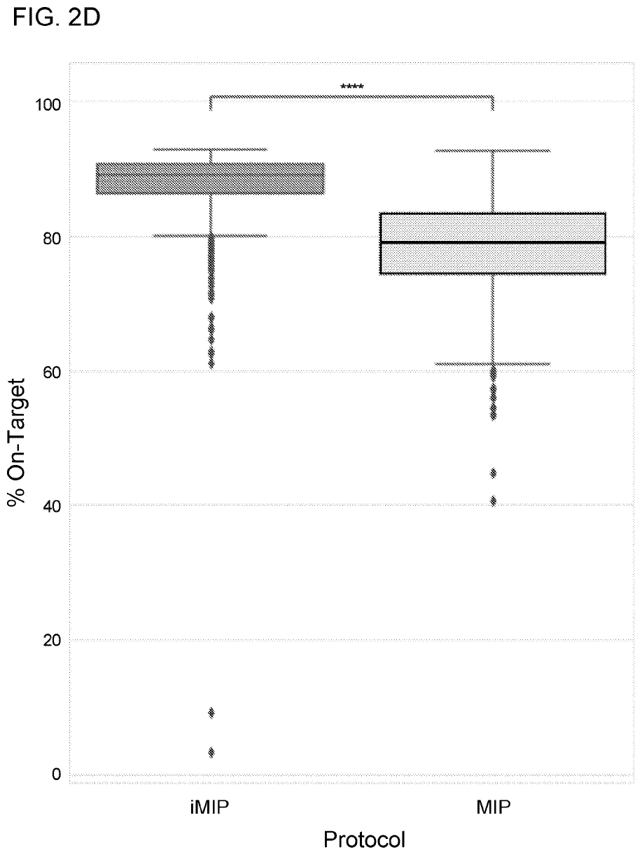

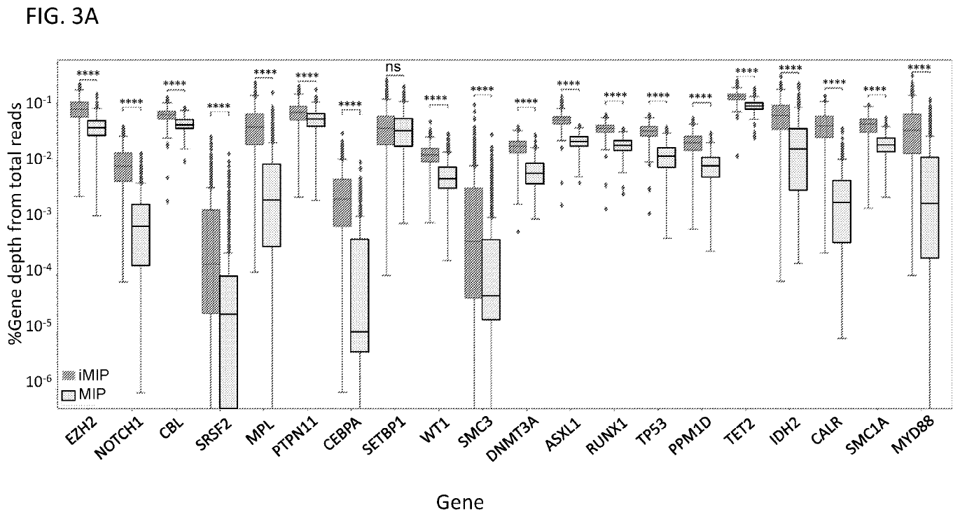

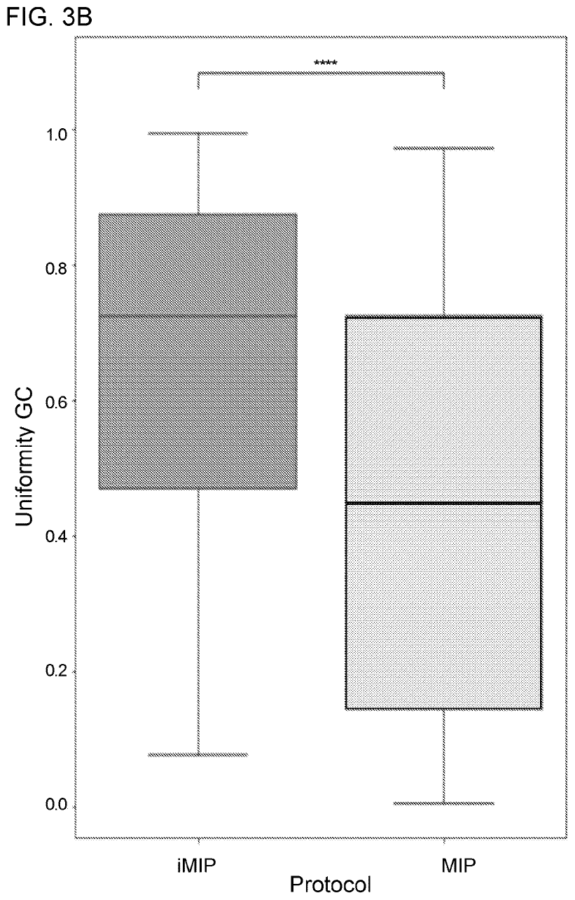

The prior art MIP protocol notoriously suffered from low: on-target %, uniformity and GC content coverage. These parameters were all significantly improved in the iMIP protocol disclosed herein (see, for example, FIGS. 2A-2D and 3A-3C).

In recent years, molecular inversion probes were used to target and sequence a variety of genomic and transcriptomic targets, e.g., the exome, short tandem repeats, disease related targets, methylation patterns and RNA expression. The iMIP protocol disclosed herein is a steppingstone towards advancing MIP library prep not just to the clinic, mainly due to the ease-of-use short turnaround time, but also to other targeted sequencing applications due to improved performance specifically in GC rich of small and medium size panels.

Thus, a first aspect of the present disclosure relates to a molecular inversion probe-based targeted sequencing method, specifically, an improved method. In some embodiments the disclosed method comprises the following steps:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. In some embodiments the hybridization of the MIP and the target nucleic acid sequence is performed in the presence of a suitable hybridization mix. In yet some further embodiments, the incubation step is performed in a thermal cycler.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. In some embodiments, such sequence synthesis is also referred to herein as a fill gap reaction. In some embodiments, at least one DNA polymerase and dNTPs are added to the hybridized MIP for performing the polymerization reaction. In some embodiments at least one ligase is added to the reaction. In yet some further embodiments, the reaction and/or ligation reaction is performed by incubating in a thermal cycler.

The next step (c) involves subjecting the reaction mixture obtained in step (b) to enzymatic digestion for 10 to 45 minutes, thereby digesting any linear MIP/s or linear nucleic acid molecule/s present in the reaction mixture.

The next step (d) involves amplifying the synthesized sequence of the cyclized product/s.

In some embodiments, the digestion step and/or the amplification step may be optional steps. Thus, in some embodiments, the disclosed method may comprise the following steps:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. In some embodiments the hybridization of the MIP and the target nucleic acid sequence is performed in the presence of a suitable hybridization mix. In yet some further embodiments, the incubation step is performed in a thermal cycler.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. In some embodiments, such sequence synthesis is also referred to herein as a fill gap reaction.

In yet some further embodiments, the disclosed methods may comprise the following steps:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. In some embodiments the hybridization of the MIP and the target nucleic acid sequence is performed in the presence of a suitable hybridization mix. In yet some further embodiments, the incubation step is performed in a thermal cycler.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. In some embodiments, such sequence synthesis is also referred to herein as a fill gap reaction.

The next step (c) involves subjecting the reaction mixture obtained in step (b) to enzymatic digestion for 10 to 45 minutes, thereby digesting any linear MIP/s or linear nucleic acid molecule/s present in the reaction mixture.

Still further optional embodiments concern methods comprising the steps of:

Step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. In some embodiments the hybridization of the MIP and the target nucleic acid sequence is performed in the presence of a suitable hybridization mix. In yet some further embodiments, the incubation step is performed in a thermal cycler.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. In some embodiments, such sequence synthesis is also referred to herein as a fill gap reaction. The next step (c) involves amplifying the synthesized sequence of the cyclized product/s.

According to some embodiments, there is provided a molecular inversion probe-based targeted sequencing method, the method comprising the steps of:

In step (a), providing at least one molecular inversion probe (MIP) comprising: (i) a first region comprising a first sequence complementary to a target nucleic acid, and (ii) a second region comprising a second sequence complementary to the target nucleic acid. The next step (b) involves contacting the at least one MIP to the target nucleic acid and a hybridization mix, and incubating in a thermal cycler for hybridization time, wherein the hybridization time is one to three and a half hours, thereby obtaining a MIP hybridized to the first and second regions of the target nucleic acid, and adding to the hybridized MIP a composition comprising dNTPs and a DNA polymerase and incubating in a thermal cycler for 1 to 20 minutes, thereby synthesizing a sequence corresponding to the target nucleic acid nested between the first and second regions of the at least one MIP. The next step (c) involves digesting the at least one MIP by enzymatic digestion for 15 to 45 minutes. The next step (d) involves amplifying the synthesized sequence. The disclosed methods provide and/or use MIPs. Molecular inversion probes (MIPs) are, e.g., nucleic acid hybridization probes that hybridize to a target nucleic acid in a loop with the 5′ and 3′ ends adjacent to or separated in the target with a small gap. The MIPs are typically designed to interrogate a target nucleotide in the gap using the high specificity of the DNA polymerase reaction. If provided with the appropriate dNTP, the polymerase can fill the gap between the MIP 5′ and 3′ ends. For example, if the target nucleic acid has an adenine “A” in the gap, using the target as a template, the polymerase can fill the gap if provided with a complementary dTTP. The polymerase will add a “T” and fill the gap in the gap-fill reaction. With the gap filled, a ligase can close the remaining nick and circularize the MIP. The circularize MIPs are then enriched or isolated. In some embodiments, because circularized single strand DNA is not a substrate for many nucleases, all other nucleic acids, including MIPs that did not hybridize and circularize (also referred to herein as linear MIPs), can be digested with one or more nuclease. MIP reaction products are typically detected after an amplification step, such as PCR using primer binding sites within the MIPs or rolling circle amplification, on a capture array.

In some embodiments, MIPs useful in the disclosed methods comprise “first” and “second” regions that comprise sequences complementary to the first and second regions, respectively, of the target nucleic acid sequence. The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 70% to 100% of the nucleotides of the other strand, with at least about 80% of the nucleotides of the other strand, specifically, about 80% to 100%, more specifically at least about 90% to 95%, and more preferably from about 98% to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. In some embodiments, homology regions of a MIP display about 100% complementarity with the corresponding complementary sequence within the target nucleic acid of interest, e.g., unless there is a mismatch at the position of the interrogated nucleotide of interest.

Still further, the complementary regions of the MIPs provided and used in the disclosed methods may be also referred to herein as homology regions. “Homology regions”, as used herein are those regions of a molecular inversion probe that are complementary to the target nucleic acid of interest. As indicated above, MIPs typically have two homology regions (HRs), one at or near the 5′ end of the probe and one at or near the 3′ end. In some embodiments, the HRs are adapted to hybridize to a target nucleic acid of interest so that they about each other or are separated by a gap of a single target nucleotide or a plurality of target nucleotides. In some embodiments, the first and second complementary region of the target nucleic acid sequence, flank the sequence to be interrogated (e.g., SNP etc.). A gap of a plurality of target nucleotides can include, e.g., from 1 to about 2000 nucleotides, for example, from 1 to 500 nucleotides, and more preferably 1 to 250 nucleotides. The size of the gap will depend on a variety of factors, including the sequence of the intended target, the size of the overall MIP, the quantity and size of non-HR portions of the MIP, the desired purpose of the assay and associated characteristics, and other factors. For instance, a MIP designed to interrogate a SNP may have a gap of a single nucleotide while a MIP designed to interrogate a multi-base insertion may have a gap of multiple nucleotides. In some embodiments, the first and/or the second homology regions of the disclosed MIP may be about 10 to about 200 nucleotides long, specifically, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 105, 110, 115, 120, 125, 130, 135, 140, 145, 150, 155, 160, 165, 170, 175, 180, 185, 190, 195, 200 or more nucleotides. It should be further noted that the first and second complementary regions of the disclosed MIPs may be either the same or different.

In some embodiments, the MIP prob used in the present disclosure may comprise degenerative homology arms, or complementary regions. In some embodiments, the complementary regions of the disclosed MIPs may comprise one or more degenerate base, specifically, between about 0.1% to about 90% degenerate bases, and are therefore referred to herein as degenerative homology regions or arms, complementary regions or arms. More specifically, degenerate base means more than one base possibility at a particular position. An oligonucleotide sequence can be synthesized with multiple bases at the same position, this is termed as degenerate base also sometime referred as “wobble” position or “mixed base”. IUB (International Union of Biochemistry) has established single letter codes for all possible degenerate possibilities. An example is “R” that is A+G at the same position with 50% of the oligo sequence will have an A at that position, and the other 50% have G. A degenerate base position may have any combination of two, three, or four bases. Chemical synthesis of oligos using IUB degenerate bases is programmed and automated to deliver the percentage of each base for reaction at that specific base position; example for the letter “N”, 25% of each base will be delivered for coupling. The delivery and coupling may not be 100% accurate and efficient for each base and thus approximately 10% deviation should be expected and considered in the final oligo sequence. For degenerate (mixed bases) positions use the following IUB codes. R=A+G, Y=C+T, M=A+C, K=G+T, S=G+C, W=A+T, H=A+T+C, B=G+T+C, D-G+A+T, V=G+A+C, N=A+C+G+T.

Still further, in some embodiments, the MIP prob used in the present disclosure may comprise additional elements, for example, identifies (UMIs), sequences complementary to primers, and the like. In some embodiments, the MIP probe may comprise one or more Unique Molecular Identifiers (UMI). UMIs, are unique molecular identifiers composed of short sequences or molecular “tags”, for the purpose of identifying the specific MIP used. In yet some further embodiments, the MIP may comprise two UMIs. Still further, the at least one UMI of the disclosed MIP prob may flank at least one of the first and second complementary region (or homology arms). In yet some further embodiments, the at least one UMI of the disclosed MIP prob may be flanked by the at least one of the first and second complementary region (or homology arms). Still further, in some embodiments, the UMI may comprise between about 5 nucleotides to about 50 nucleotides, specifically, between 5 to 40 nucleotides, between 5 to 40 nucleotides, between 5 to 40 nucleotides, between 5 to 40 nucleotides, specifically, 5, 6, 7, 8, 9, 10 nucleotides. In some embodiments UMIs useful in the disclosed MIPs comprise 7 nucleotides. In yet some further embodiments, UMIs useful in the disclosed MIPs comprise 8 nucleotides. The term “flanked” as used herein refers to a nucleic acid sequence positioned between two defined regions.

Step (a) of the disclosed methods involves hybridization of the target nucleic acid sequence with the at least one MIPs. The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” Hybridizations are usually performed under stringent conditions, for example, at a temperature of at least 25° C. and more. As other factors may affect the stringency of hybridization, including base composition and length of the complementary strands, presence of organic solvents and extent of base mismatching, the combination of parameters is more important than the absolute measure of any one alone. The hybridization step of the disclosed methods is performed in conditions suitable to allow the successful hybridization of the at least one MIP to the target sequence, thereby forming the hybridized MIP. In some embodiments, “hybridizing conditions” include any condition (time, temperature, buffer) that result in specific hybridization between complementary sequences, e.g., target nucleic acid sequence is said to specifically hybridize to the MIP probe nucleic acid complementary region when it hybridizes at least 50% as well (e.g., quantitatively under the same hybridization conditions) to the probe as to the perfectly matched complementary target, i.e., with a signal to noise ratio at least half as high as hybridization of the probe to the target under conditions in which the perfectly matched probe binds to the perfectly matched complementary target.

More specifically, in some embodiments, step (a) of the disclosed methods is performed in the presence of a suitable hybridization buffer. The hybridization buffer may comprise in some embodiments ampligase reaction buffer. More specifically, in some embodiments, the 10× Ampligase Reaction Buffer comprises 200 mM Tris-HCl (pH 8.3), 250 mM KCl, 100 mM MgCl2, 5 mM NAD, and 0.1% Triton® X-100. In some embodiments appropriate concentration of the buffer is used. More specifically, the hybridization mixture may comprise between about 2× to about 0.1× ampligase reaction buffer, specifically, between about 0.1× to about 1× of the ampligase reaction buffer specified herein. More specifically, about 0.1×, 0.2×, 0.3×, 0.4×, 0.5×, 0.6×, 0.7×, 0.8×, 0.9×, 1× or less. In yet some further embodiments, the final concentration of the ampligase reaction buffer in the hybridization mixture is about 0.80×, 0.81×, 0.82×, 0.83×, 0.84×, 0.85×, 0.86×, 0.87×, 0.88×, 0.89×, 0.9×, more specifically, about 0.85× ampligase reaction buffer. Thus, in some embodiments, 0.85× Ampligase Reaction Buffer is used. In some embodiments, the hybridization step may be performed in an appropriate temperature that allows denaturation of the target nucleic acid sequence and/or the MIP prob, into single strands followed by annealing of the complementary region of the probe to the corresponding complementary region in the target nucleic acid sequence. In some embodiments the denaturation may be performed in a high temperature for a suitable period of time. Hybridization mixture as used herein, is meant in some embodiments, a mixture that comprises the hybridization buffer as specified above, the at least one MIP/s and the target nucleic acid sequence. Non-limiting embodiments therefore include incubation of the hybridization mixture that contains the target sequence and the at least one MIP/s at a temperature of between about 90° C. to about 100° C. or more, specifically, a temperature of about 90° C., 91° C., 92° C., 93° C., 94° C., 95° C., 96° C., 97° C., 98° C., 99° C., 100° C., or more, specifically, 98° C., for a suitable time period. For example, between bout 0.1 min to about 10 minutes, specifically, about 0.5 minute, 1, 2, 3, 4, 5 minutes or more, specifically, for 3 minutes. Thus, in some embodiments, the hybridization mixture is incubated for 3 minutes at 98° C. In some embodiments, following the denaturation in 98° C., the hybridization mixture is further incubated at appropriate temperature for an appropriate time period, for example, at a temperature of between about 60° C. to about 100° C. or more, specifically, at 75° C., 76° C., 77° C., 78° C., 79° C., 80° C., 81° C., 82° C., 83° C., 84° C., 85° C., 86° C., 87° C., 88° C., 89° C., 90° C. or more, specifically, at 85° C., for a suitable time period. More specifically, for between bout 0.1 min to about 60 minutes, specifically, for about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 minutes, or more. In some embodiments, the mixture is incubated at 85° C., for 30 minutes. In yet some further embodiments, the mixture is incubated at 85° C., for 20 minutes. Still further, annealing of the complementary sequences may be performed in some embodiments at a temperature of between about 30° C. to about 80° C. or more, specifically, a temperature of about 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C. or more, specifically, at 60° C. for a suitable period of time. For example, for between bout 0.1 min to about 200 minutes, about 1 minute to about 200 minutes, specifically, for about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 51, 52, 53, 54, 55, 56, 57, 58, 59, 60, 61, 62, 63, 64, 65, 66, 67, 68, 69, 70, 75, 80, 85, 90, 95, 100, 105, 110, 115, 120, 125, 130, 135, 140, 145, 150 minutes or more. In some specific embodiments, for about 60 minutes. In yet some further embodiments, for 40 minutes. Thus, in some embodiments, the hybridization mixture is incubated at 60° C. for 60 minutes, or alternatively, at 60° C. for 40 minutes. Still further, in some embodiments, this step is followed by a further incubation at a temperature of between about 30° C. to about 80° C. or more, specifically, a temperature of 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C. or more, specifically, at 56° C., for a suitable period of time. For example, for between bout 0.1 min to about 200 minutes, specifically, for about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, 90, 95, 100, 105, 110, 115, 120, 125, 130, 135, 140, 145, 150 minutes or more. In some specific embodiments, for about 60 minutes. In yet some further embodiments, for 40 minutes. Thus, in some embodiments, the hybridization mixture is incubated at 56° C. for 60 minutes, or alternatively, at 56° C. for 40 minutes. In some embodiments, the reaction is kept in 56° C. until the polymerization reaction starts. Thus, in some embodiments, this step may involve incubation at 98° C. for about 3 minutes, followed by 85° C. for about 30 minutes or less, then 60° C. for about 60 minutes or less, and 56° C., for about 60 minutes or less. In yet some alternative embodiments, the hybridization step comprises incubation of the hybridization mixture at 98° C. for about 3 minutes, followed by 85° C. for about 20 minutes or less, then 60° C. for about 40 minutes or less, and 56° C., for about 40 minutes or less.

In some embodiments, this step may be performed in a thermal cycler. In yet some further embodiments, the hybridization program used may be either gradual (ramp temp) or constant. Thermocycler (also known as a thermal cycler, PCR machine or DNA amplifier), as used herein, is a laboratory apparatus most commonly used to amplify segments of DNA via the polymerase chain reaction (PCR). Thermal cyclers may also be used in laboratories to facilitate other temperature-sensitive reactions, including enzymatic reaction (polymerization, exonuclease, restriction enzyme digestion, ligation). The device has a thermal block with holes where tubes holding the reaction mixtures can be inserted. The cycler then raises and lowers the temperature of the block in discrete, pre-programmed steps. The ramp rate of a thermal cycler indicates the change in temperature from one PCR step to another over time and is usually expressed in degrees Celsius per second (° C./sec). The terms “up ramp” and “down ramp” refer to the heating and cooling of thermal blocks, respectively.

In yet some further embodiments, in step (b) of the disclosed methods, polymerization and/or ligation are performed. Thus, as indicated above, in some embodiments the polymerization and ligation (b), involves subjecting the hybridized MIP obtained in step (a), to a polymerization reaction for 1 to 20 minutes, thereby synthesizing a sequence corresponding to the target nucleic acid sequence nested between the first and second regions of the at least one MIP. In some embodiments, such sequence synthesis is also referred to herein as a fill gap reaction. A “gap-fill reaction” is a reaction, described herein, in which a gap is filled by the action of a polymerase between 5′ and 3′ ends of a molecular inversion probe hybridized to a complementary target nucleic acid. In many embodiments, the filled gap consists of a single nucleotide. However, in some MIP gap-fill reactions the gap can be more than one nucleotide, for example, between about 1 to about 500 nucleotides, specifically, between about 1 to about 450 nucleotides, between about 1 to about 400 nucleotides, between about 1 to about 350 nucleotides, between about 1 to about 300 nucleotides, between about 1 to about 250 nucleotides, e.g., 2, 3, 4, 5, 6, 7, 8, 9, 10, 15, 20, 25, 30, 40, 50 75, 100, 150, 200, 250 or more nucleotides, e.g., between first and second MIP homology regions specifically hybridized to a target nucleic acid. In some embodiments, the methods disclosed herein may further encompass gaps of hundreds of nucleotides, and/or gaps between different chromosomes, that may be used in methods that define genomic topological organization, as will be discussed in more detail herein after. It should be understood that the synthesized sequence is further ligated to obtain cyclized product/s in the polymerization and/or ligation reaction mixture.

In some embodiments, the polymerization reaction is performed by a DNA polymerase. A polymerase as used herein, is a member of a group of enzymes required for DNA synthesis. The main function of the DNA polymerase is to synthesize DNA during replication. DNA polymerase works in pairs, replicating two strands of DNA in tandem. They add deoxyribonucleotides at the 3′-OH group of the growing DNA strand. The DNA strand grows in 5′→3′ direction by their polymerization activity. Adenine pairs with thymine and guanine pairs with cytosine. DNA polymerases cannot initiate the replication process and they need a primer to add to the nucleotides. The polymerization reaction is therefore the synthesis of the DNA strand that corresponds to the appropriate template, as indicated above in connection with the gap-fill reaction.

There are five DNA polymerases identified in E. coli. All the DNA polymerases differ in structure, functions and rate of polymerization and processivity. DNA Polymerase I is coded by polA gene. It is a single polypeptide and has a role in recombination and repair. It has both 5′→3′ and 3′→5′ exonuclease activity. DNA polymerase I removes the RNA primer from lagging strand by 5′→3′ exonuclease activity and also fills the gap. DNA Polymerase II is coded by polB gene. It is made up of 7 subunits. Its main role is in repair and also a backup of DNA polymerase III. It has 3′→5′ exonuclease activity. DNA Polymerase III is the main enzyme for replication in E. coli. It is coded by polC gene. It also has proofreading 3′→5′ exonuclease activity. DNA Polymerase IV is coded by dinB gene. Its main role is in DNA repair during SOS response, when DNA replication is stalled at the replication fork.

According to some embodiments, the DNA polymerase may be any DNA polymerase known in the art. According to some embodiments, the DNA polymerase is a high-fidelity DNA polymerase. High-Fidelity DNA Polymerase sets a new standard for both fidelity and robust performance. With the highest fidelity amplification available (˜280 times higher than Taq), Q5 DNA Polymerase results in ultra-low error rates. Q5 DNA Polymerase is composed of a novel polymerase that is fused to the processivity-enhancing Sso7d DNA binding domain, improving speed, fidelity and reliability of performance. According to some embodiments, the high-fidelity DNA polymerase in GC enriched DNA regions. According to some embodiments, the DNA polymerase includes, but is not limited to, any one or more of the following: Q5 High-Fidelity (HF) DNA Polymerase, Advantage® GC Genomic LA Polymerase (Takara), PrimeSTAR® GXL DNA Polymerase (Takara) and AccuPrime™ GC-Rich DNA Polymerase (Invitrogen), Platinum SuperFi II DNA Polymerase (Thermo Fisher Scientific), KAPA2G Robust HotStart PCR Kit. Still further, in some specific embodiments, a Q5 high fidelity DNA polymerase is used in the present polymerization reaction.

In some embodiments, at least one DNA polymerase and dNTPs are added to the hybridized MIP for performing the polymerization reaction.

More specifically, in some embodiments, the reaction mixture as referred to herein may comprise in some embodiments any suitable elements required for the polymerization reaction. More specifically, in some embodiments, a polymerization reaction is performed using a polymerization reaction buffer. In some embodiments the polymerization reaction buffer may comprise at least one of Q5 High GC Enhancer, beta-nicotinamide adenine dinucleotide (NAD+), dNTPs, betaine, and an appropriate DNA polymerase. In some embodiments, the reaction mixture used in the disclosed methods may comprise dNTPs (e.g., 14 pM), Betaine (e.g., 375 mM), NAD+ (e.g., 1 mM), additional Ampligase buffer as specified above, for example, between about 0.1× to about 1× of the ampligase reaction buffer as specified herein. More specifically, about 0.1×, 0.2×, 0.3×, 0.4×, 0.5×, 0.6×, 0.7×, 0.8×, 0.9×, 1× or less. In yet some further embodiments, the final concentration of the ampligase reaction buffer in the polymerization mixture is about 0.450×, 0.46×, 0.47×, 0.48×, 0.49×, 0.50×, 0.51×, 0.52×, 0.53×, 0.54×, 0.55×, more specifically, about 0.50× ampligase reaction buffer, Ampligase (e.g., total of 1.25 U) and Q5 High-Fidelity DNA Polymerase (e.g., 0.4 U). In yet some alternative or additional embodiments, the polymerization reaction may comprise a “Q5 Reaction Buffer”. In some embodiments, a 5×Q5 Reaction Buffer may comprise 2 mM Mg++ at final (1×) reaction concentrations. Thus, in some embodiments, the Q5 Reaction Buffer is between about 0.1× to about 1×, specifically, about 0.1×, 0.2×, 0.3×, 0.4×, 0.5×, 0.6×, 0.7×, 0.8×, 0.9×, 1× or less.

In yet some further embodiments, the final concentration of the Q5 Reaction Buffer in the polymerization mixture is about 0.150×, 0.16×, 0.17×, 0.18×, 0.19×, 0.20×, 0.21×, 0.22×, 0.23×, 0.24×, 0.25×, 0.26×, 0.27×, 0.28×, 0.29×, 0.30×, 0.31×, 0.32×, 0.33×, 0.34×, 0.35×, more specifically, about 0.250× ampligase reaction buffer. Thus, in some alternative or additional embodiments, the reaction mixture may further comprise Q5 reaction buffer (e.g., 0.25×). Still further, for GC-rich targets (≥65% GC), amplification can be improved by the addition of the 5×Q5 High GC Enhancer, as indicated above.

In some embodiments at least one ligase is added to the reaction. In yet some further embodiments, the reaction and/or ligation reaction is performed by incubating in a thermal cycler. More specifically, DNA Ligase, as used herein, is an enzyme that catalyzes the NAD-dependent ligation of adjacent 3′-hydroxyl and 5′-phosphate termini in duplex DNA structures. Derived from a thermophilic bacterium, Ampligase DNA Ligase is stable and active at much higher temperatures than conventional DNA ligases. The half-life of Ampligase DNA Ligase is 48 hours at 65° C. and more than 1 hour at 95° C. In most cases, the upper limit on reaction temperatures with Ampligase DNA Ligase is determined by the Tm of the DNA substrate. Under conditions of maximal hybridization stringency, nonspecific ligation is nearly eliminated. Ampligase DNA Ligase has no detectable activity on blunt ends or RNA substrates. The enzyme is active in a variety of DNA polymerase buffers within a pH range of 7-8. It should be understood that any ligase may be used for the disclosed method.

Still further, in some embodiments, the polymerization and ligation step (b) may be performed at an appropriate temperature for a suitable period of time. More specifically, in some embodiments, the hybridized MIP products obtained in step (a) are incubated at a temperature of between about 30° C. to about 100° C. or more, specifically, a temperature of 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C. or more, specifically, at 56° C., for a suitable period of time. In some embodiments, the suitable incubation time may be 0.5, 1, 2, 3, 4, 5, 6, 7, 8, 9, 10 or more minutes, specifically, 5 minutes. In some particular embodiments, the reaction mixture is incubated for 5 minutes at 56° C. In some embodiments, the incubation in 56° C. is followed by additional incubation at a suitable temperature, for example a temperature of between about 30° C. to about 100° C. or more, specifically, 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 73° C., 74° C., 85° C., or more, specifically, at 72° C., for a suitable period of time. In some embodiments, the suitable incubation time may be between about 0.1 min to about 30 minutes, specifically, 0.5, 1, 2, 3, 4, 5, 6, 7, 8, 9, 10 or more minutes, specifically, 5 minutes. Thus, in some embodiments, the reaction mixture is incubated at 56° C. for 5 minutes followed by 72° C. for 5 minutes. It should be understood that in some embodiments where the target nucleic acid sequence is an RNA, prior to the hybridization reaction, the nucleic acid molecules are converted into DNA molecules, specifically, cDNA molecules by reversed transcription, for example by using reverse transcriptase.

Still further, in some embodiments, for each of the specified reaction steps upon ending the reaction as specified above, and before proceeding to the next step, the reaction may be kept in cold, for example, between 4° C. to 20° C., specifically at 16° C. As indicated above, in some embodiments, the disclosed methods may further comprise the optional step of amplifying the cyclized products of the polymerization reaction of step (b), or if digestion and thus enrichment of the cyclized product is performed by the of step enzymatic digestion (c), the cyclized product obtained by step (c) is amplified by any suitable amplification methods. In some particular and non-limiting embodiments, the amplification is performed using a PCR reaction.

“Polymerase chain reaction,” or “PCR,” means a reaction for the in vitro amplification of specific DNA sequences by the simultaneous primer extension of complementary strands of DNA, as is notoriously well known in the art. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art. For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g., 200 nL, to a few hundred uL, e.g., 200 uL. “Reverse transcription PCR,” or “RT-PCR,” means a PCR that is preceded by a reverse transcription reaction that converts a target RNA to a complementary single stranded DNA, which is then amplified. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences are simultaneously carried out in the same reaction mixture.

In some embodiments, the hybridization time is less than three and a half hours.

In yet some further embodiments, the hybridization time is one to three hours.

Still further, in some embodiments, the hybridization time is one to two and a half hours.

In some embodiments, the hybridization time is between 60 to 200 minutes, specifically, 60, 65, 70, 75, 80, 85, 90, 95, 100, 101, 102, 103, 104, 105, 110, 115, 120, 125, 130, 135, 140 145, 150, 155, 160, 165, 170, 175, 180, 185, 190, 195 or 200 minutes. In some embodiments the hybridization time is 150 minutes or less, in yet some alternative embodiments, the hybridization time is 135 minutes or less, in some further embodiments, hybridization time is 120 minutes or less, in some further embodiments, the hybridization time is 103 minutes or less.

As indicated above, for separating the cyclized products obtained in the polymerization and ligation step (b) from any linear MIPs or other linear nucleic acid molecules that may be present in the reaction mixture, the disclosed method may optionally comprise an addition step of enzymatic digestion. However, it should be appreciated that in some embodiments, the digestion involves the use of at least one exonuclease. The term “Exonucleases” refers to enzymes that catalyze the removal of nucleotides in either the 5-prime to 3-prime or the 3-prime to 5-prime direction from the ends of single-stranded and/or double-stranded DNA. Removal of nucleotides is achieved by cleavage of phosphodiester bonds via hydrolysis. Most exonucleases digest at nicks in the DNA. Some exonucleases remove one base at a time. Lambda Exonuclease is an example of this and transforms double-stranded DNA into single-stranded DNA by chewing from the free ending containing a 5-prime phosphate, degrading one strand preferentially but not the other. Other examples are Exo I and Exo III. Other exonucleases, such as T5, ExoV or Exo VII remove short oligos. The products of T5 Exo also include individual bases. Exonucleases such as Exo VII and V, digest in both the 5-prime to 3-prime and 3-prime to 5-prime direction, while others, such as Exo T and Exo I, only work in one direction. Some exonucleases, such as Exo I and Exo T only digest single-stranded DNA while leaving behind double-stranded DNA. Exonucleases such as T7 Exo digest only double-stranded DNA, while others, such as T5 Exo and Exo V, can digest both single and double-stranded DNA. In more specific embodiments, Exonuclease I and/or Exonuclease III are used. In some embodiments, any form of linear MIP probe and/or nucleic acid sequence is removed following the gap-fill reaction by digestion with a combination of exonucleases. The exonuclease mixture contains exonuclease I and exonuclease III. Exonuclease I may digest single-stranded DNA in a 3′→5′ direction, requires a free 3′-hydroxyl terminus, but does not digest double-stranded DNA. Exonuclease III is a 3′-exonuclease which catalyzes the removal of mononucleotides from the 3′-OH end of double stranded DNA. It also dephosphorylates DNA strands which possess a 3′-phosphate group and has RNase H activity. Exonuclease VII digests DNA from free 3′ or 5′ ends. Exonuclease VII has been reported to have little activity on circularized DNA.

In some embodiments, the digestion reaction is performed by adding Exonuclease I and/or Exonuclease III to the reaction mixture of step (b) and incubation at an appropriate temperature, for a suitable period of time. In some embodiments, the digestion reaction is performed at 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., or more. In some embodiments, the reaction is incubated at 37° C. for a suitable period of time. In some embodiments, the incubation time is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 minutes or more, specifically, 10 minutes. In some embodiments the digestion reaction is performed at 37° C. for 10 minutes. Still further, the digestion reaction is followed by inactivation of the nucleases. This step is performed at a suitable temperature for a suitable period of time. More specifically, at 65° C., 66° C., 67° C., 68° C., 69° C., 70° C., 71° C., 72° C., 73° C., 48° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., or more, specifically, any one of 80° C., 90° C. or 95° C. In some embodiments, the inactivation step may be performed for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 minutes or more. In some embodiments, the inactivation step may be for 20 minutes. In yet some further embodiments, the inactivation step may last 5 minutes.

Still further, in some embodiments, the digestion reaction is performed by incubation of the mixture of step (b) at 37° C. for 10 minutes. Still further, this step is followed by inactivation of the exonucleases at 80° C. for 20 minutes. In yet some further embodiments, the digestion step is performed by incubation of the mixture with the disclosed exonucleases at 37° C. for 10 minutes followed by inactivation for 5 minutes at 90° C. or 95° C.

In some embodiments, the entire process that includes steps (a) to (c) of the disclosed methods is performed within less than 200 minutes. More specifically, 200 minutes or less, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100 minutes or less. In some embodiments, the hybridization time is 153 minutes, the polymerization time is 10 minutes, and the digestion time is 30 minutes or 15 minutes, thereby, all three steps may be performed within 193 to 178 minutes. In some embodiments within 193 or 187 minutes. Still further in some embodiments the hybridization time is 135 minutes, the polymerization time is 10 minutes, and the digestion time is 30 minutes or 15 minutes, thereby, all three steps may be performed within 175 to 160 minutes. In some embodiments within 175 or 160 minutes.

In some embodiments, the hybridization time is 120 minutes, the polymerization time is 10 minutes, and the digestion time is 30 minutes or 15 minutes, thereby, all three steps may be performed within 160 to 145 minutes. In some embodiments within 160 or 145 minutes. Still further, in some embodiments, the hybridization time is 103 minutes, the polymerization time is 10 minutes, and the digestion time is 30 minutes or 15 minutes, thereby, all three steps may be performed within 143 to 138 minutes. In some embodiments within 143 or 138 minutes. According to some embodiments, the at least one MIP comprises a plurality of MIPs corresponding to a plurality of different target regions. The term “plurality” as used herein refers to more than one. More specifically, the disclosed method may use 1 to 100,000 or more different MIPs directed either to the same or to a different target nucleic acid sequence. For example, 1 to 90,000, 1 to 85,000, 1 to 80,000, 1 to 75,000, 1 to 70,000, 1 to 65,000, 1 to 60,000, 1 to 55,000, 1 to 50,000, 1 to 45,000, 1 to 40,000, 1 to 35,000, 1 to 30,000, 1 to 25,000, 1 to 20,000, 1 to 15,000, 1 to 10,000, 1 to 900, 1 to 9000, 1 to 8500, 1 to 8000, 1 to 7500, 1 to 7000, 1 to 6500, 1 to 6000, 1 to 5500, 1 to 5000, 1 to 4500, 1 to 4000, 1 to 3500, 1 to 3000, 1 to 2500, 1 to 2000, 1 to 1500, 1 to 1000, 1 to 950, 1 to 900, 1 to 850, 1 to 800, 1 to 750, 1 to 700, 1 to 650, 1 to 600, 1 to 550, 1 to 500, 1 to 450, 1 to 400, 1 to 350, 1 to 300, 1 to 250, 1 to 200, 1 to 150, 1 to 100, 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, 1 to 10, specifically, 1, 2, 3, 4, 5, 6, 7, 8, 9, 10, 15, 20, 25, 30, 40, 50 75, 100, 150, 200, 250, 500, 1000, 10,000, 100,000 or more MIPs.

In yet some further embodiments, the disclosed method further comprise sequencing a plurality of synthesized sequences obtained in step (d) and identifying variants of interest.

Thus, the disclosed method may further comprise in some embodiments thereof, an additional step of sequencing. More specifically, the synthetized sequences obtained by the disclosed methods are subjected in some optional embodiments to any suitable sequencing method. Sequencing of the target sequence thus allows to define various variants of the analyzed target sequence. DNA sequencing is the process of determining the nucleic acid sequence—the order of nucleotides in DNA. It includes any method or technology that is used to determine the order of the four bases: adenine, guanine, cytosine, and thymine. Several methods for DNA sequencing were developed and became commercially available in the past two decades. Together these were called the “next-generation” or “second-generation” sequencing (NGS) methods, in order to distinguish them from the earlier methods, including Sanger sequencing. NGS technology is typically characterized by being highly scalable, allowing the entire genome to be sequenced at once. Usually, this is accomplished by fragmenting the genome into small pieces, randomly sampling for a fragment, and sequencing it using one of a variety of technologies. An entire genome sequencing is possible because multiple fragments are sequenced at once (giving it the name “massively parallel” sequencing) in an automated process. More specifically, NGS generates large quantities of sequence data within a shorter time duration and massive cost reduction as compared to conventional Sanger's sequencing method. This technique uses different chemistries, matrices and bioinformatics technologies which can be used to sequence entire in shorter time periods. DNA sequencing pipeline includes various steps which includes, DNA fragmentation, NGS Library preparation (these two can be combined by transposase mediated library preparation) Sequencing and Data analysis. In DNA Fragmentation, targeted DNA is broken into several small segments using different methods like sonication and enzymatic digestion. The next step involves the preparation of a NGS Library, wherein each piece of the fragmented DNA is modified DNA to be sequencing ready, namely by adding DNA sequences (adapters) that are required for sequencing instrument compatibility, in some embodiments of DNA sequencing generally termed “targeted sequencing” the desired target is captured after library preparation (“probe capture” or amplified “amplicon/MIP” from the genomic template. In the latter, the required DNA sequences are attached after amplification as described above or during the amplification protocol. The library is sequenced using the various DNA sequencing methods. Each DNA fragment has an adapter on one end that connects it to a solid substrate such as beads or flow cells, and another adapter on the other end that anneals to a primer that starts the polymerase chain reaction (PCR). PCR produces several copies of the same fragment, which are sequenced at the same time. As a result, these techniques are sometimes referred to as massively parallel sequencing techniques. DNA Sequencing may be performed in some embodiments, using an NGS sequencer. In a specific sequencer, the library is uploaded onto a sequencing matrix. The platform on which the sequencing takes place is known as a sequencing matrix. Sequencing matrices differ depending on the sequencer. For example, the Illumina NGS sequencer uses flow cells, while the Ion torrent NGS sequencer uses sequencing chips.

Several generations of sequencing methods have been developed. The present disclosure encompasses the use of any known method. To name but few, Pyrosequencing/454 Sequencing, ABI SOLID, Solexa/Illumina Sequencing, Pacific Biosciences Single Molecule Real Time Reads, Nanopore DNA Sequencing, Singular Genomics G4, Element Biosciences AVITI, Ultima Genomics.

The required short segments are isolated using different methods such as Hybridization Capture Assay, Amplicon Assay.

Still further, in some embodiments, the disclosed method may further comprise applying machine learning algorithm on the identified variants or a subgroup thereof, for calculating sensitivity, specificity and precision thereof.

In some embodiments, the subgroup of variants comprises variants having VAF below threshold. The present disclosure thus provides a sensitive and improved method displaying noise reduction allowing detection of variants with VAF as low as 10%, 9%, 8%, 7%, 6%, 5%, 4%, 3%, 2%, 1%, 0.1%, 0.9%, 0.8%, 0.7%, 0.6%, 0.5%, 0.4%, 0.3%, 0.2%, 0.1%, more specifically, between 0.5% to 0.6%, specifically, 0.51%, 0.52%, 0.53%, 0.54%, 0.55%, 0.56%, 0.57%, 0.58%, 0.59%, 0.6%, or less, specifically, 0.5, with sensitivity of about 100% to 75%, specifically, 100%, 99%, 98%, 97%, 96%, 95%, 94%, 93%, 92%, 91%, 90%, 89%, 88%, 87%, 86%, 85%, 84%, 3%, 82%, 81%, 80%, 79%, 78%, 77%, 76%, 75%, or less, and specifically, 80% sensitivity and significantly higher precision.

It should be noted that the at least one MIP used by the disclosed method may be a double strand probe. However, it should be appreciated that also single strand MIPs may be applicable in the disclosed methods.

It should be appreciated that in some embodiments the target nucleic acid sequence may be any genomic nucleic acid sequence. In some embodiments, genomic nucleic acid sequence may include nuclear DNA and non-nuclear DNA or may be any either linear or circular nucleic acids. For example, nuclear DNA, specifically, chromosomal DNA and Microbiome DNA (e.g., Gut microbiome), as well as circular genomic DNA such as mitochondrial DNA and chloroplast DNA (cpDNA). Still further, genomic nucleic acid sequence may further include genomic nucleic acid molecules of any organism or microorganism as disclosed in the present disclosure, or any nucleic acid sequence of any infectious entity, for example, viruses, specifically, any viruses disclosed by the present disclosure, or any bacteriophages and transducing particles. In some embodiments, the target nucleic acid sequences may be of chromosomal or non-chromosomal source. Nucleic acid sequences of non-chromosomal source encompassed by the present disclosure include transposons, plasmids, mitochondrial DNA, and chloroplast DNA, as well as nucleic acid molecules of any other genetic element. Still further, in some embodiments, the target nucleic acid sequence applicable in the disclosed methods may be any circulating free DNA (cfDNA). More specifically, Cell-free nucleic acids (cf-NAs) include several types of DNA (cf-DNA) and RNA molecules (cell-free non-coding RNAs, and protein coding RNA—mRNA) that are present in extracellular fluids. There are two main types of cf-DNA: cell-free nuclear DNA (cf-nDNA) and cell-free mitochondrial DNA (cf-mtDNA). More specifically, Circulating free DNA (cfDNA) are degraded DNA fragments of about 50 to 200 bp, that are released to the blood plasma. cfDNA can be used to describe various forms of DNA freely circulating in the bloodstream, including circulating tumor DNA (ctDNA), cell-free mitochondrial DNA (ccf mtDNA), and cell-free fetal DNA (cffDNA). Still further, the target nucleic acid sequence applicable in the methods of the present disclosure may be in some embodiments, cell free non-coding RNA or long non-coding RNAs. More specifically, Cell free non-coding RNA (cf-ncRNAs) relate to small non-coding RNA, including but not limited to microRNAs (miRNA), siRNA, piRNA, snRNA, snoRNA, YRNA etc, or long non-coding RNA (lncRNAs) including but not limited to pseudogen RNA, telomerase RNA, circular RNA (cirRNA), etc.

Long non-coding RNAs (lncRNAs) as used herein, are non-protein-coding transcripts with a length of more than 200 nt. They can be transcribed from intergenic regions (long intervening non-coding RNAs), from the introns of protein-coding genes (intronic lncRNAs) or as antisense transcripts of genes. They have broad molecular functions: they may be involved in the epigenetic regulation of allelic expression (e.g., in X chromosome dosage compensation in female mammals), they may act as scaffolds for protein complexes or as decoys for specific target molecules to limit their availability (e.g., lncRNAs possess binding sites for miRNAs, regulating their abundance). They may also serve as precursors for small non-coding RNAs (sncRNA) or be involved in post-transcriptional gene regulation (e.g., antisense lncRNAs binding to their corresponding sense transcripts and alter splice-site recognition or spliceosome recruitment in mRNA processing). In yet some further embodiments, the target sequence may be transcriptomic nucleic acid sequence, thereby providing information with respect to the transcriptome and/or the exome of an organism.

The term “target nucleic acid of interest”, as used herein, refers to the sample nucleic acid putatively including a target sequence of interest. The target sequence of interest, with regard to a MIP includes those sequences complementary to the MIP homology regions. The sequence may include one or more interrogated nucleotides that may or may not match a corresponding nucleotide on a MIP homology region, or may or may not provide a substrate for a polymerase provided with the complementary dNTP/s.

Still further, the terms “target nucleic acid sequence of interest”, “nucleic acid sequence of interest”, “a target gene of interest”, “a target gene”, are used interchangeably, and refer in some embodiments to a nucleic acid sequence that may comprise or comprised within a gene or any fragment or derivative thereof. The target nucleic acid sequence or gene of interest may comprise coding or non-coding DNA regions, or any combination thereof. In some embodiments, the nucleic acid sequence of interest may comprise coding sequences and thus may comprise exons or fragments thereof that encode any product. In other embodiments, the target nucleic acid sequence of interest may comprise non-coding sequences, as for example start codons, 5′ un-translated regions (5′ UTR), 3′ un-translated regions (3′ UTR), or other regulatory sequences, in particular regulatory sequences.

In some embodiments, the target gene or nucleic acid sequence of interest may be any nucleic acid sequence or gene or fragments thereof that display aberrant expression, stability, activity or function in a mammalian subject, as compared to normal and/or healthy subject. Such target gene or any fragments thereof or any target nucleic acid sequence may be in some embodiments, associated, linked or connected, directly or indirectly with at least one pathologic condition. More specifically, the length of the nucleic acid sequence of interest may be about 100,000 nucleotides in length, or less than 75,000 nucleotides in length or less than 50,000 nucleotides in length, or less than 40,000 nucleotides in length, or less than 30,000 nucleotides in length, or less than 20,000 nucleotides in length, or less than 15,000 nucleotides in length, or less than 10,000 nucleotides in length, or less than 5000 nucleotides in length, or less than 1000 nucleotides in length, or less than 900 nucleotides in length, or less than 800 nucleotides in length, or less than 700 nucleotides in length, or less than 600 nucleotides in length, or less than 500 nucleotides in length, or less than 450 nucleotides in length, or less than 400 nucleotides in length, or less than 300 nucleotides in length, or less than 200 nucleotides in length, or less than 100 nucleotides in length, or less than 50 nucleotides in length, or less than 40 nucleotides in length, or less than 30 nucleotides in length, or less than 20 nucleotides in length, or less than 10 nucleotides in length.

The disclosed methods provide effective approach for sequencing target nucleic acid sequences. The term “nucleic acid molecule or sequence” is referred to often herein, and relates to DNA, RNA, single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence and any chemical modifications thereof. Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine, and isoguanidine and the like. Modifications can also include 3′ and 5′ modifications such as capping.

In some embodiments, the target nucleic acid sequence is a nucleic acid sequence associated with, or comprising, at least one of: genetic and/or epigenetic variation/s, pathologic disorder/s, infectious entity, e.g., pathogenic entity, microorganism/s and GC-rich regions.

In some embodiments, the target nucleic acid sequence may comprise or is associated with genetic or epigenetic variations that may be associated with pathologic disorders. It is understood that the interchangeably used terms “associated”, “linked” and “related”, when referring to pathologies as disclosed herein after, mean any genetic or epigenetic variations which at least one of: cause either directly or indirectly, responsible for, share causalities, co-exist at a higher than coincidental frequency, with at least one disease, disorder condition or pathology or any symptoms thereof. In yet some further embodiments, the target nucleic acid sequence may either be associated with or comprising nucleic acid sequence of infectious entity, for example, a pathogenic entity. Infectious entities and specifically pathogenic entities, for example, viruses, parasites, bacteria, fungi, and the like, are encompassed by the present aspect, are disclosed herein after.

In some embodiments, the disclosed MIP-based targeted sequencing methods are particularly useful for target nucleic acid sequences comprising GC-rich regions. As indicated herein, the disclosed methods are particularly effective and applicable for target nucleic acid sequences that comprise GC-regions or display high GC-content. GC-content (or guanine-cytosine content) is the percentage of nitrogenous bases in a DNA or RNA molecule that are either guanine (G) or cytosine (C). This measure indicates the proportion of G and C bases out of an implied four total bases, also including adenine and thymine in DNA and adenine and uracil in RNA.

GC-content may be given for a certain fragment of DNA or RNA or for an entire genome. When it refers to a fragment, it may denote the GC-content of an individual gene or section of a gene (domain), a group of genes or gene clusters, a non-coding region, or a synthetic oligonucleotide such as a primer. The GC content of a gene region can impact its coverage, with regions having 50-60% GC content receiving the highest coverage while regions with high (70-80%) or low (30-40%) GC content having significantly decreased coverage.

In more specific embodiments, genetic variations comprise at least one of: single nucleotide variant (SNVs) and/or single-nucleotide polymorphisms (SNPs), insertions and/or deletions, (indels), inversions, copy number variations (CNV), loss of heterozygosity (LOH), gene fusions, translocations, duplications, structural variants, alternative splicing, and variable number of tandem repeats.

The term “single nucleotide polymorphism” (SNP) as herein defined, refers to a single base change in the DNA sequence. For a base position with sequence alternatives in genomic DNA to be considered as a SNP, the least frequent allele (the “minor allele”) should have a frequency of 1% or greater. The most frequent allele is referred to as the “major allele”. SNPs are usually bi-allelic, mainly due to the low frequency of single nucleotide substitutions in DNA. As known to a person skilled in the art, the term “SNP” usually refers to the least frequent allele (i.e. the minor allele), when present in the genome either on both chromosomes (then an individual is said to be homozygous for a certain polymorphism) or on a single chromosome (then an individual is said to be heterozygous for a certain polymorphism). Known specific SNPs are assigned with unique identifiers, usually referred to by accession numbers with a prefix such as “SNP”, “refSNP” or “rs”, as known to one of skill in the art. Single nucleotide polymorphism database (dbSNP) of nucleotide sequence variation is available on the NCBI website.

Copy-number variation, as used herein, is meant variation from one person to another in the number of copies of a particular gene or DNA sequence.

Deletion, refers to any mutation that involves the loss of genetic material. It can be small, involving a single missing DNA base pair, or large, involving hundreds or thousands of nucleotides, and in some embodiments event a piece of a chromosome.

Indel as referred to herein relates to an insertion or deletion of bases in the genome of an organism. It is classified among small genetic variations, measuring from 1 to 10,000 base pairs in length. A microindel is defined as an indel that results in a net change of 1 to 50 nucleotides.

Insertion mutation, as used herein is a mutation involving the addition of genetic material. An insertion mutation can be small, involving a single extra DNA base pair, or large, involving a piece of a chromosome/s.

Inversion, is a chromosomal segment that has been broken off and reinserted in the same locus, but with the reverse orientation.

Translocation refers to herein as the positional change of one or more chromosome segments in cells or gametes.

Still further, in some embodiments, the disclosed methods may be applicable for determining and identifying structural variations in nucleic acid molecules, for example, genomic organization or topological organization of nucleic acids. More specifically, although genomes are defined by their sequence, the linear arrangement of nucleotides is only their most basic feature. A fundamental property of genomes is their topological organization in three-dimensional space in the intact cell nucleus. The application of imaging methods and genome-wide biochemical approaches, combined with functional data, is revealing the precise nature of genome topology/organization and its regulatory functions in gene expression and genome maintenance. In the context of the subject disclosure, genomic organization refers to the linear order of DNA elements and their division into chromosomes. Genome organization can also refer to the 3D structure of chromosomes and the positioning of DNA sequences within the nucleus. There are several techniques to capture chromosome/chromatin confirmation. One non-limiting example for high-throughput genomic and epigenomic technique to capture chromatin conformation is the Hi-C (or standard Hi-C) technique. In general, Hi-C is considered as a derivative of a series of chromosome conformation capture technologies, including but not limited to 3C (chromosome conformation capture), 4C (chromosome conformation capture-on-chip/circular chromosome conformation capture), and 5C (chromosome conformation capture carbon copy). Hi-C comprehensively detects genome-wide chromatin interactions in the cell nucleus by combining 3C and next-generation sequencing (NGS) approaches and has been considered as a qualitative leap in C-technology (chromosome conformation capture-based technologies) development and the beginning of 3D genomics.

Still further, the disclosed methods may be applicable in detecting epigenetic modifications. Epigenetics as referred to herein, relates to heritable phenotype changes that do not involve alterations in the nucleic acid sequence. Epigenetics most often involves changes that affect gene activity and expression, and thereby the phenotype of the cell. Epigenetic modifications or variations, involve in some embodiments, covalent modification of the DNA sequence or of proteins associated with DNA organization and functioning. In some embodiments, epigenetic variations as disclosed herein comprise DNA methylation, (e.g. cytosine methylation and hydroxymethylation), histone modifications (e.g. lysine acetylation, lysine and arginine methylation, serine and threonine phosphorylation, and lysine ubiquitination and sumoylation). In some embodiments, the methods disclosed herein may be useful for interrogating DNA methylation degree, and pattern. DNA methylation is a stable, heritable, covalent modification to DNA, occurring mainly at CpG dinucleotides, but is also found at non-CpG sites. Methylation is associated with normal developmental processes, as well as the changes that are observable during oncogenesis and other pathological processes, such as gene silencing of tumor suppressor or DNA repair genes. Bisulfite genomic sequencing is regarded as a gold-standard technology for detection of DNA methylation and provides a qualitative, quantitative and efficient approach to identify 5-methylcytosine at single base-pair resolution. This method is based on the finding that the amination reactions of cytosine and 5-methylcytosine (5mC) proceed with very different consequences after the treatment of sodium bisulfite. The MIP based sequencing methods of the present disclosure may be therefore applicable in identifying epigenetic modifications.

Still further, in some embodiments, the target nucleic acid sequence is associated with at least one hereditary, somatic, congenital, spontaneous, or acquired pathologic disorder or condition. The term “Hereditary disease” as herein defined refers to a disease or disorder that is caused by defective genes which are inherited from the parents. A hereditary disease may result unexpectedly when two healthy carriers of a defective recessive gene reproduce but can also happen when the defective gene is dominant. Non-limiting examples of hereditary diseases include Duchenne muscular dystrophy (DMD), Cystic Fibrosis, Tay-Sachs disease (also known as GM2 gangliosidosis or hexosaminidase A deficiency), Ataxia-Telangiectasia (A-T), Sickle-cell disease (SCD), or sickle-cell anemia (SCA or anemia), Lesch-Nyhan syndrome (LNS, also known as Nyhan's syndrome, Amyotrophic Lateral Sclerosis, Cystinosis, Kelley-Seegmiller syndrome and Juvenile gout), color blindness, Haemochromatosis (or haemosiderosis), Haemophilia, Phenylketonuria (PKU), Phenylalanine Hydroxylase Deficiency disease, Polycystic kidney disease (PKD or PCKD, also known as polycystic kidney syndrome), Alpha-galactosidase A deficiency, Fabry disease, Anderson-Fabry disease, Angiokeratoma Corporis Diffusum, CADASIL (cerebral autosomal dominant arteriopathy with subcortical infarcts and leukoencephalopathy), Cerebral arteriopathy with subcortical infarcts and leukoencephalopathy, Cerebral autosomal dominant ateriopathy with subcortical infarcts and leukoencephalopathy, Carboxylase Deficiency, Multiple (Late-Onset), Cerebroside Lipidosis syndrome, Gaucher's disease, Choreoathetosis self-mutilation hyperuricemia syndrome, Classic Galactosemia, Galactosemia, Crohn's disease, also known as Crohn syndrome and regional enteritis, Incontinentia Pigmenti (also known as “Bloch-Siemens syndrome,” “Bloch-Sulzberger disease,” “Bloch-Sulzberger syndrome” “melanoblastosis cutis,” and “naevus pigmentosus systematicus”), galactosemia Microcephaly, alpha-1 antitrypsin deficiency (Alpha-1), Adenosine deaminase (ADA) deficiency, Severe Combined Immunodeficiency (SCID), neurofibromatosis type 1 (NF1), Wiskott-Aldrich syndrome, Stargardt macular degeneration, Fanconi's anemia, Spinal muscular atrophy (SMA) and Leber's congenital amaurosis (LCA).

In yet some further embodiments, the disorders may be congenital disorders. More specifically, A congenital disorder is a medical condition that is present at or before birth. These conditions, also referred to as birth defects, can be acquired during the fetal stage of development or from the genetic make up of the parents. Congenital disorders are not necessarily hereditary, since they may be caused by infections during pregnancy or injury to the fetus at birth. Major anomalies are sometimes associated with minor anomalies, which might be objective (e.g., preauricular tags) or more subjective (e.g. low-set ears). Non limiting embodiments include external disorders and internal disorders such as Neural tube defects, Microcephaly, Microtia/Anotia, Orofacial clefts, Exomphalos (omphalocele), Gastroschisis, Hypospadias, Reduction defects of upper and lower limbs, Talipes, equinovarus/club foot, Congenital heart defects, Esophageal atresia/tracheoesophageal fistula, Large intestinal atresia/stenosis, Anorectal atresia/stenosis and Renal agenesis/hypoplasia.

Still further, in some embodiments, the disorders may be somatic disorders. A somatic symptom disorder, formerly known as a somatoform disorder is any mental disorder that manifests as physical symptoms that suggest illness or injury, but cannot be explained fully by a general medical condition or by the direct effect of a substance, and are not attributable to another mental disorder (e.g., panic disorder). Somatic symptom disorders, as a group, are included in a number of diagnostic schemes of mental illness. Somatic disorders may be also referred to as somatization disorder and undifferentiated somatoform disorder.

In yet some further embodiments pathologic disorders applicable in the present disclosure my be any spontaneous, or acquired pathologic disorder, for example, and disorder caused by environmental exposure to a pathogenic agent or any environmental stress or condition.

In yet some further embodiments, the pathologic disorder may be at least one of: a proliferative disorder, and/or a neoplastic disorder, a metabolic condition, an inflammatory disorder, an infectious disease caused by a pathogen, a mental disorder, an autoimmune disease, a cardiovascular disease, a neurodegenerative disorder, fetal genetic condition and an age-related condition. Still further, pathologic disorders encompassed by the present disclosure further include infections and parasitic diseases, endocrine, nutritional diseases, immunity disorders, diseases of blood and blood forming organs, mental disorders, diseases of nervous system and sense organs, diseases of the circulatory system, diseases of the respiratory system, diseases of the digestive system, diseases of genitourinary system, complications of pregnancy, childbirth and the puerperium, diseases of the skin and subcutaneous tissue, diseases of musculoskeletal system and connective tissue and congenital anomalies.

In yet some further embodiments, the methods of the present disclosure may be applicable for any neoplastic disorder and/or any proliferative disorder. More specifically, as used herein to describe the present disclosure, “neoplastic disorder”, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present disclosure may be applicable for diagnosing of a patient suffering from any one of non-solid and solid tumors. Malignancy, as contemplated in the present disclosure may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present disclosure can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The disclosed methods may be applicable for solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. In yet some further embodiments, the methods of the present disclosure may be applicable for any of the proliferative disorders discussed herein. In more specific and non-limiting embodiments, the methods of the present disclosure may be specifically applicable for at least one of non-small cell lung cancer (NSCLC) melanoma, renal cell cancer, ovarian carcinoma and breast carcinoma.

Still further, it should be appreciated that the methods disclosed herein are applicable for any neoplastic disorder, specifically, any malignant or non-malignant proliferative disorder. In yet some further embodiments, the method and uses of the present disclosure are applicable for any cancer. Thus, in some illustrative and non-limiting embodiments, the methods and uses of the present disclosure may be applicable for any one of: Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma-see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer). In some specific and non-limiting embodiments, the target sequence is associated with age related condition. In more specific embodiments, the age-related disorder may be age-related clonal hematopoiesis (ARCH). Accordingly, the target nucleic acid sequence is a sequence associated with ARCH.

In more particular embodiments, such target sequences may be any sequence comprised within the CCAAT Enhancer Binding Protein Alpha (CEBPA) gene (HGNC: 1833). In yet some further particular and non-limiting embodiments, the target sequences may be any sequence comprised within the SET binding protein 1 (SETBP1) gene (HGNC: 15573).

In some embodiments, the at least one target nucleic acid sequence is derived from a genomic DNA of a human subject prone to have ARCH. Age-related clonal hematopoiesis (ARCH) is defined as the gradual, clonal expansion of hematopoietic stem and progenitor cells (HSPCs) carrying specific, disruptive, and recurrent genetic variants, in individuals without clear diagnosis of hematological malignancies. ARCH is associated not just with chronological aging but also with several other, age-related pathological conditions, including inflammation, vascular diseases, cancer mortality, and high risk for hematological malignancies. Although it remains unclear whether ARCH is a marker of aging or plays an active role in these various pathophysiologies, it is suggested here that treating or even preventing ARCH may prove to be beneficial for human health (Shlush L I. Age-related clonal hematopoiesis. Blood. 2018 Feb. 1; 131(5):496-504).

A further aspect of the present disclosure relates to a method for diagnosing a pathological disorder in a subject by identifying at least one genetic and/or epigenetic variation/s and/or at least one nucleic acid sequence of at least one pathogenic entity associated with the pathologic disorder in at least one target nucleic acid sequence of at least one sample of the subject. More specifically, the method comprising the step of performing molecular inversion probe-based targeted sequencing in at least one test sample of the subject or in any nucleic acid molecule obtained therefrom. It should be understood that the presence of one or more of the variation/s in at least one target nucleic acid sequence, and/or the presence of at least one nucleic acid sequence of at least one pathogenic entity in the examined sample indicates that the subject has a risk, is a carrier, or is suffering from the pathologic disorder. In some embodiments, the molecular inversion probe-based targeted sequencing method performed herein comprises the following steps.

One step (a) involves contacting at least one molecular inversion probe (MIP) with at least one target nucleic acid sequence of the subject that may contain the genetic variation associated with the disorder and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

In some embodiments, the molecular inversion probe-based targeted sequencing method is performed in the disclosed diagnostic method as defined by the present disclosure.

More specifically, in some embodiments, the hybridization time of the MIP-based targeted sequencing method used by the disclosed diagnostic methods is less than three and a half hours. In yet some further embodiments, the hybridization time of the MIP-based targeted sequencing method used by the disclosed diagnostic methods is one to three hours.

Still further, in some embodiments, the hybridization time of the MIP-based targeted sequencing method used by the disclosed diagnostic methods is one to two and a half hours.

Still further, in some embodiments, the step of enzymatic digestion of all linear MIPs and/or nucleic acid molecules that may be present in the reaction mixture obtained in step (b) of the MIP-based targeted sequencing method used by the disclosed diagnostic methods, may last for about 15 to 30 minutes.

In some embodiments, the entire process that includes steps (a) to (c) of the of the MIP-based targeted sequencing method used by the disclosed diagnostic methods is performed within less than 200 minutes. In some embodiments, the hybridization time is 153 minutes, the polymerization time is 10 minutes, and the digestion time is 30 minutes or 15 minutes, thereby, all three steps may be performed within 193 to 178 minutes. In some embodiments, 193 or 178 minutes. Still further in some embodiments the hybridization time is 135 minutes, the polymerization time is 10 minutes, and the digestion time is 30 minutes or 15 minutes, thereby, all three steps may be performed within 175 to 160 minutes. In some embodiments, 175 or 160 minutes.

In some embodiments, the hybridization time is 120 minutes, the polymerization time is 10 minutes, and the digestion time is 30 minutes or 15 minutes, thereby, all three steps may be performed within 160 to 145 minutes. In some embodiments, 160 or 145 minutes. Still further, in some embodiments, the hybridization time is 103 minutes, the polymerization time is 10 minutes, and the digestion time is 30 minutes or 15 minutes, thereby, all three steps may be performed within 143 to 138 minutes. In some embodiments, 143 or 138 minutes.

In some embodiments, the MIP-based targeted sequencing method used by the disclosed diagnostic methods may use at least one MIP, specifically, a plurality of MIPs corresponding or targeted at, or specific for to a plurality of different target regions.

In yet some further embodiments, the MIP-based targeted sequencing method used by the disclosed diagnostic methods may further comprise sequencing a plurality of synthesized sequences obtained in step (d) and identifying variants of interest.

Still further, in some embodiments, the MIP-based targeted sequencing method used by the disclosed diagnostic methods may further comprise applying machine learning algorithm on the identified variants or a subgroup thereof, for calculating sensitivity, specificity and precision thereof. In some embodiments, the subgroup of variants comprises variants having VAF below threshold.

It should be noted that the at least one MIP used by the MIP-based targeted sequencing method used by the disclosed diagnostic methods, may be a double strand probe. However, it should be appreciated that also single strand MIPs may be applicable in the disclosed methods.

It should be appreciated that in some embodiments the target nucleic acid sequence used in the diagnostic methods, may be any genomic nucleic acid sequence. In yet some further embodiments, the target sequence may be transcriptomic nucleic acid sequence, thereby providing information with respect to the transcriptome and/or the exome of an organism.

In some embodiments, the target nucleic acid sequence is a nucleic acid sequence associated with, or comprising, at least one of: genetic variation/s, pathologic disorder/s, pathogenic entity, microorganism/s and GC-rich regions.

In some embodiments the diagnostic methods disclosed herein are applicable for any subject. Such subject may be at least one organism of the biological kingdom Animalia or at least one organism of the biological kingdom Plantae.

Thus, the methods of the present disclosure may be applicable for any subject of the biological kingdom Animalia. It should be understood that an organism of the Animalia kingdom in accordance with the present disclosure includes any invertebrate or vertebrate organism.

More specifically, Invertebrates are animals that neither possess nor develop a vertebral column (commonly known as a backbone or spine), derived from the notochord. This includes all animals apart from the subphylum Vertebrata. More specifically, invertebrates include the Phylum Porifera—Sponges, the Phylum Cnidaria—Jellyfish, hydras, sea anemones, corals, the Phylum Ctenophora—Comb jellies, the Phylum Platyhelminthes-Flatworms, the Phylum Mollusca-Molluscs, the Phylum Arthropoda-Arthropods, the Phylum Annelida-Segmented worms like earthworm and the Phylum Echinodermata-Echinoderms. Familiar examples of invertebrates include insects; crabs, lobsters and their kin; snails, clams, octopuses and their kin; starfish, sea-urchins and their kin; jellyfish and worms.

Still further, in some embodiments, the methods of the present disclosure may be applicable for a vertebrate organism. Vertebrates comprise all species of animals within the subphylum Vertebrata (chordates with backbones). The animals of the vertebrates group include Fish, Amphibians, Reptiles, Birds and Mammals (e.g., Marsupials, Primates, Rodents and Cetaceans).

Vertebrates represent the overwhelming majority of the phylum Chordata, with currently about 66,000 species described. Vertebrates include the jawless fish and the jawed vertebrates, which include the cartilaginous fish (sharks, rays, and ratfish) and the bony fish.

Still further, in some embodiments, the subject of the present disclosure may be any one of a human or non-human mammal, an avian, an insect, a fish, an amphibian, a reptile, a crustacean, a crab, a lobster, a snail, a clam, an octopus, a starfish, a sea-urchin, jellyfish, and worms. In more specific embodiments, the subject referred to herein may be a mammal. In yet some further embodiments, such mammalian organisms may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks).

In yet some further embodiments, the present disclosure may be applicable for any organism of the order primates. More specifically, primates are divided into two distinct suborders, the first is the strepsirrhines that includes lemurs, galagos, and lorisids. The second is haplorhines—that includes tarsier, monkey, and ape clades, the last of these including humans. In yet some further embodiments, the present disclosure may be applicable for any organism of the subfamily Homininae, that includes the hylobatidae (gibbons) and the hominidae that includes ponqunae (orangutans) and homininae [gorillini (gorilla) and hominini ((panina (chimpanzees) and hominina (humans))].

In some specific embodiment, the methods of the present disclosure may be applicable for a mammal that may be any domestic mammal, for example, at least one of a Cattle, domestic pig (swine, hog), sheep, horse, goat, alpaca, lama and Camels. Still further, in some embodiments, the mammalian subject is human subject.

As mentioned above, the present disclosure concerns any eukaryotic organism and as such, may be also applicable for members of the biological kingdom Plantae.

In more specific embodiments, the disclosed methods may be applicable for any plant. In more specific embodiments, such plant may be a dioecious plant or monoecious plant.

More specifically, in some embodiments the organism of the biological kingdom Plantae may be a dioecious plant, specifically, a plant presenting biparental reproduction. In some specific embodiments, the plant diagnosed by the disclosed methods may be of the family Cannabaceae, specifically, any one of Cannabis (hemp, marijuana) and Humulus (hops). In more specific embodiments, the plant of the family Cannabaceae may be Cannabis (hemp, marijuana). In yet some further embodiments, the plant of the family Cannabaceae may be Humulus (hops).

In some embodiments, any plants are applicable in the present disclosure, for example, any model plants such as, Arabidopsis, Tobacco, Solanum licopersicum, Solanum tuberosum.

In yet some further embodiments, Canola, Cereals (Corn wheat, Barley), rice, sugarcane, Beet, Cotton, Banana, Cassava, sweet potato, lentils, chickpea, peas, Soy, nuts, peanuts, Lemna, Apple, may be applicable in the present disclosure.

A non-comprehensive list of useful annual and perennial, domesticated or wild, monocotyledonous or dicotyledonous land plant or Algae—(i.e unicellular or multicellular algae including diatoms, microalgae, ulva, nori, gracilaria), applicable in accordance with the present disclosure may include but are not limited to crops, ornamentals, herbs (i.e., labiacea such as sage, basil and mint, or lemon grass, chives), grasses (i.e., lawn and biofuel grasses and animal feed grasses), cereals (i.e., rice, wheat, rye, oats, corn), legumes (i.e. soy, beans, lentils, chick peas, peas, peanuts), leafy vegetables (i.e. kale, bok-choi, cress, lettuce, spinach, cabbage), Amaranthacea (i.e. sugar beet, beet, quinoa, spinach), Compositea (i.e. sunflower, lettuce, aster), Malvaceae (i.e. cotton, cacao, okra, hibiscus), cucurbits (i.e., cucumber, squash, melon, watermelon), Solanaceous species (i.e tobacco, potato, tomato, petunia and pepper), Umbellifera (i.e. carrot, celery, dill, parsley, cumin), Crucifera (i.e., oilseed rape, mustard, brassicas, cauliflower, radish), Sesame, the monocot Aspargales (i.e. onion, garlic, leek, asparagus, vanilla, lilies, tulips, narcissus), Myrtacea (i.e., Eucalyptus, pomegranate, guava), Subtropical fruit trees (i.e. Avocado, Mango, Litchi, papaya), Citrus (i.e. orange, lemon, grapefruit), Rosacea (i.e. apple, cherry, plum, almond, roses), berry-plants (i.e. grapes, mulberries, blueberries, raspberry, strawberry), nut trees (i.e. macademia, hazelnut, pecan, walnut, chestnuts, brazil nut, cashew), banana and plantain, palms (i.e., oil-palm, coconut and dates), evergreen, coniferous or deciduous trees, woody species.

In some embodiments, the diagnostic methods of the present disclosure may detect at least one nucleic acid sequence of a pathogenic entity associated with a pathologic disorder in a subject. In some embodiments, such pathogenic entity is at least one of a viral, a bacterial, a fungal, a parasitic and a protozoan pathogen, as defined by the present disclosure.

Still further, in some embodiments, the genetic variations that are associated with the diagnosed pathologic disorder comprise at least one of: single nucleotide variant (SNVs) and/or single-nucleotide polymorphisms (SNPs), insertions and/or deletions, (indels), inversions, copy number variations (CNV), loss of heterozygosity (LOH), structural variations, gene fusions, translocations, duplications, variable number of tandem repeats, as defined in connection with other aspects of the present disclosure.

In some embodiments, the target nucleic acid sequence analyzed by the disclosed diagnostic methods is associated with at least one congenital, hereditary, somatic, spontaneous, or acquired pathologic disorder or condition. Specifically, any of the disorders defined in connection with other aspects of the present disclosure.

Still further, in some embodiments, the diagnostic methods disclosed herein may be applicable for any pathologic disorder. Such pathologic disorder is at least one of: a proliferative disorder, a metabolic condition, an inflammatory disorder, an infectious disease caused by a pathogen, an autoimmune disease, a cardiovascular disease, a neurodegenerative disorder, fetal genetic condition and an age-related condition. Still further, pathologic disorders encompassed by the present disclosure further include infections and parasitic diseases, endocrine, nutritional diseases, immunity disorders, diseases of blood and blood forming organs, mental disorders, diseases of nervous system and sense organs, diseases of the circulatory system, diseases of the respiratory system, diseases of the digestive system, diseases of genitourinary system, complications of pregnancy, childbirth and the puerperium, diseases of the skin and subcutaneous tissue, diseases of musculoskeletal system and connective tissue and congenital anomalies.

In some embodiments, the diagnostic methods disclosed herein may be applicable for any age-related condition. In more specific embodiments, the diagnostic method disclosed herein are applicable for diagnosing ARCH in a subject. In some embodiments, the disclosed diagnostic methods are applicable for a human subject prone to have ARCH.

A further aspect of the present disclosure relates to a method of detecting the presence of one or more target microorganism or infectious entity, (e.g., pathogenic or non-pathogenic entity) in a test sample. More specifically, the method comprising the step of performing molecular inversion probe-based targeted sequencing in at least one nucleic acid molecule obtained from the sample. It should be noted that the presence of one or more target nucleic acid sequence associated with the microorganism or infectious entity in the sample indicates the presence thereof in the sample. In some embodiments, the molecular inversion probe-based targeted sequencing method applicable in the disclosed detection methods, comprising the step of:

One step (a) involves contacting at least one nucleic acid molecule of the sample with at least one MIP specific for at least one target nucleic acid sequence associated with the microorganism or infectious entity and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

In some embodiments, the molecular inversion probe-based targeted sequencing method may be performed in the disclosed microorganism, infectious entity, or pathogen-detecting method, as defined by the present disclosure. More specifically, in some embodiments, the hybridization time of the MIP-based targeted sequencing method used by the disclosed microorganism or pathogen-detecting methods is less than three and a half hours. In yet some further embodiments, the hybridization time of the MIP-based targeted sequencing method used by the disclosed microorganism or pathogen-detecting methods is one to three hours. Still further, in some embodiments, the hybridization time of the MIP-based targeted sequencing method used by the disclosed microorganism or pathogen-detecting methods is one to two and a half hours. Still further, in some embodiments, the step of enzymatic digestion of all linear MIPs and/or nucleic acid molecules that may be present in the reaction mixture obtained in step (b) of the MIP-based targeted sequencing method used by the disclosed microorganism or pathogen-detecting methods, may last for about 15 to 30 minutes.

In some embodiments, the entire process that includes steps (a) to (c) of the of the MIP-based targeted sequencing method used by the disclosed microorganism or pathogen-detecting methods is performed within less than 200 minutes. In some embodiments, the hybridization time is 153 minutes, the polymerization time is 10 minutes, and the digestion time is 30 minutes or 15 minutes, thereby, all three steps may be performed within 193 to 178 minutes. In some embodiments, 193 or 178 minutes. Still further in some embodiments the hybridization time is 135 minutes, the polymerization time is 10 minutes, and the digestion time is 30 minutes or 15 minutes, thereby, all three steps may be performed within 175 to 160 minutes. In some embodiments, 175 or 160 minutes.

In some embodiments, the hybridization time is 120 minutes, the polymerization time is 10 minutes, and the digestion time is 30 minutes or 15 minutes, thereby, all three steps may be performed within 160 to 145 minutes. In some embodiments, 160 or 145 minutes. Still further, in some embodiments, the hybridization time is 103 minutes, the polymerization time is 10 minutes, and the digestion time is 30 minutes or 15 minutes, thereby, all three steps may be performed within 143 to 138 minutes. In some embodiments, 143 or 138 minutes.

In some embodiments, the MIP-based targeted sequencing method used by the disclosed microorganism or pathogen-detecting methods may use at least one MIP, specifically, a plurality of MIPs corresponding or targeted at, or specific for to a plurality of different target regions.

In yet some further embodiments, the MIP-based targeted sequencing method used by the disclosed microorganism or pathogen-detecting methods may further comprise sequencing a plurality of synthesized sequences obtained in step (d) and identifying variants of interest.

Still further, in some embodiments, the MIP-based targeted sequencing method used by the disclosed microorganism, infectious entity, or pathogen-detecting methods, may further comprise applying machine learning algorithm on the identified variants or a subgroup thereof, for calculating sensitivity, specificity and precision thereof. In some embodiments, the subgroup of variants comprises variants having VAF below threshold.

It should be noted that the at least one MIP used by the MIP-based targeted sequencing method used by the disclosed microorganism or pathogen-detecting methods, may be a double strand probe. However, it should be appreciated that also single strand MIPs may be applicable in the disclosed methods.

In some embodiments the target nucleic acid sequence used in the microorganism or pathogen-detecting methods, may be any genomic nucleic acid sequence. In yet some further embodiments, the target sequence may be transcriptomic nucleic acid sequence. In yet some further embodiments, the target sequence may be any circulating nucleic molecule as disclosed by the present disclosure. Still further the target sequence may be any of the nucleic acid molecules as defined in connection with other aspects disclosed herein.

In some embodiments, the target nucleic acid sequence is a nucleic acid sequence associated with, or comprising, at least one of: genetic variation/s, pathologic disorder/s, pathogenic entity, microorganism/s and GC-rich regions.

In some embodiments, the microorganism detected by the disclosed methods is a prokaryotic microorganism, or a lower eukaryotic microorganism. In yet some further embodiments, the infectious entity, for example, the pathogenic entity detected by the disclosed methods is at least one of a viral, a bacterial, a fungal, a parasitic and a protozoan pathogen.

As used herein, the term “pathogen” refers to an infectious agent that causes a disease in a subject host. Pathogenic agents include prokaryotic microorganisms, lower eukaryotic microorganisms, complex eukaryotic organisms, viruses, fungi, mycoplasma, prions, parasites, for example, a parasitic protozoan, yeasts or a nematode.

In yet some further embodiments, the methods of the present disclosure may be applicable for detecting a pathogen that may be in further specific embodiment, a viral pathogen or a virus. In some embodiments, the pathogen may be at least one viral pathogen.

The term “virus” as used herein, refers to obligate intracellular parasites of living but non-cellular nature, consisting of DNA or RNA and a protein coat. Viruses range in diameter from about 20 to about 300 nm. Class I viruses (Baltimore classification) have a double-stranded DNA as their genome; Class II viruses have a single-stranded DNA as their genome; Class III viruses have a double-stranded RNA as their genome; Class IV viruses have a positive single-stranded RNA as their genome, the genome itself acting as mRNA; Class V viruses have a negative single-stranded RNA as their genome used as a template for mRNA synthesis; and Class VI viruses have a positive single-stranded RNA genome but with a DNA intermediate not only in replication but also in mRNA synthesis.

It should be noted that the term “viruses” is used in its broadest sense to include any virus, specifically, any enveloped virus. In some specific embodiments, the viral pathogen may be of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages).

In some embodiments, the viral pathogens applicable in the disclosed methods may be DNA viruses, specifically, any virus of the following families: the Adenoviridae family, the Papovaviridae family, the Parvoviridae family, the Herpesviridae family, the Poxviridae family, the Hepadnaviridae family and the Anelloviridae family.

In yet some further specific embodiments, the viral pathogens applicable in the disclosed methods may be RNA viruses, specifically, any virus of the following families: the Reoviridae family, Picornaviridae family, Caliciviridae family, Togaviridae family, Arenaviridae family, Flaviviridae family, Orthomyxoviridae family, Paramyxoviridae family, Bunyaviridae family, Rhabdoviridae family, Filoviridae family, Coronaviridae family, Astroviridae family, Bornaviridae family, Arteriviridae family, Hepeviridae family and the Retroviridae family. Of particular interest are viruses of the families adenoviruses, papovaviruses, herpesviruses: simplex, varicella-zoster, Epstein-Barr (EBV), Cytomegalo virus (CMV), pox viruses: smallpox, vaccinia, hepatitis B (HBV), rhinoviruses, hepatitis A (HBA), poliovirus, respiratory syncytial virus (RSV), Middle East Respiratory Syndrome (MERS-COV), Severe acute respiratory syndrome (SARS-Cov), SARS-COV2, corona virus, rubella virus, hepatitis C (HBC), arboviruses, rabies virus, influenza viruses A and B, measles virus, mumps virus, human deficiency virus (HIV), HTLV I and II and Zika virus.

In some specific and embodiments, the methods of the present disclosure may be suitable for detecting at least one corona virus (CoV). CoVs are common in humans and usually cause mild to moderate upper-respiratory tract illnesses. There are four main sub-groupings of coronaviruses, known as alpha, beta, gamma, and delta. The seven coronaviruses known to-date as infecting humans are: alpha coronaviruses 229E and NL63, and beta coronaviruses OC43, HKU1, SARS-COV and SARS-COV2, and MERS-COV (the coronavirus that causes Middle East Respiratory Syndrome, or MERS). The SARS-COV and SARS-COV2 are a lineage B beta Coronavirus and the MERS-COV is a lineage C beta Coronavirus.

Still further, in some embodiments, the disclosed methods may be applicable for detecting bacteria, and in some embodiments, bacterial pathogens. The term “bacteria” (in singular a “bacterium”) in this context refers to any type of a single celled microbe. Herein the terms “bacterium” and “microbe” are interchangeable. This term encompasses herein bacteria belonging to general classes according to their basic shapes, namely spherical (cocci), rod (bacilli), spiral (spirilla), comma (vibrios) or corkscrew (spirochaetes), as well as bacteria that exist as single cells, in pairs, chains or clusters. It should be noted that the term “bacteria” as used herein refers to any of the prokaryotic microorganisms that exist as a single cell or in a cluster or aggregate of single cells. In more specific embodiments, the term “bacteria” specifically refers to Gram positive, Gram negative or Acid-fast organisms. The Gram-positive bacteria can be recognized as retaining the crystal violet stain used in the Gram staining method of bacterial differentiation, and therefore appear to be purple-colored under a microscope. The Gram-negative bacteria do not retain the crystal violet, making positive identification possible. In other words, the term ‘bacteria’ applies herein to bacteria with a thicker peptidoglycan layer in the cell wall outside the cell membrane (Gram-positive), and to bacteria with a thin peptidoglycan layer of their cell wall that is sandwiched between an inner cytoplasmic cell membrane and a bacterial outer membrane (Gram-negative). This term further applies to some bacteria, such as Deinococcus, which stain Gram-positive due to the presence of a thick peptidoglycan layer, but also possess an outer cell membrane, and thus suggested as intermediates in the transition between monoderm (Gram-positive) and diderm (Gram-negative) bacteria. Acid fast organisms like Mycobacterium contain large amounts of lipid substances within their cell walls called mycolic acids that resist staining by conventional methods such as a Gram stain.

In some embodiments, a pathogen to be detected by the disclosed methods, may be any bacteria involved in nosocomial infections or any mixture of such bacteria. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality and pose a significant economic burden. In view of the rising levels of antibiotic resistance and the increasing severity of illness of hospital in-patients, this problem needs an urgent solution. Common nosocomial organisms include Clostridium difficile, methicillin-resistant Staphylococcus aureus, coagulase-negative Staphylococci, vancomycin-resistant Enteroccocci, resistant Enterobacteriaceae, Pseudomonas aeruginosa, Acinetobacter and Stenotrophomonas maltophilia.

The nosocomial-infection pathogens could be subdivided into Gram-positive bacteria (Staphylococcus aureus, Coagulase-negative staphylococci), Gram-positive cocci (Enterococcus faecalis and Enterococcus faecium), Gram-negative rod-shaped organisms (Klebsiella pneumonia, Klebsiella oxytoca, Escherichia coli, Proteus aeruginosa, Serratia spp.), Gram-negative bacilli (Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). Among many others Pseudomonas aeruginosa is an extremely important nosocomial Gram-negative aerobic rod pathogen.

In some embodiments, the disclosed methods may be applicable in detecting “ESKAPE” pathogens. As indicated herein, these pathogens include but are not limited to Enterococcus faecium, Staphylococcus aureus, Clostidium difficile, Klebsiella pneumoniae, Acinetobacter baumanii, Pseudomonas aeruginosa, and Enterobacter.

In further embodiments the pathogen according to the present disclosure may be a bacterial cell of at least one of E. coli, Pseudomonas spp, specifically, Pseudomonas aeruginosa, Staphylococcus spp, specifically, Staphylococcus aureus, Streptococcus spp, specifically, Streptococcus pyogenes, Salmonella spp, Shigella spp, Clostidium spp, specifically, Clostridium difficile, Enterococcus spp, specifically, Enterococcus faecium, Klebsiella spp, specifically, Klebsiella pneumonia, Acinetobacter spp, specifically, Acinetobacter baumanni, Yersinia spp, specifically, Yersinia pestis and Enterobacter species or any mutant, variant isolate or any combination thereof.

A lower eukaryotic organism applicable in the present invention disclosure may include in some embodiments, a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the disclosed methods.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

Still further, in certain embodiments the methods of the present disclosure may be suitable for detecting fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous and possess branched somatic structures (hyphae) surrounded by cell walls containing glucan or chitin or both and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present disclosure also provides methods that may be suitable for detecting a parasitic pathogen. More specifically, “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species.

As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

In some embodiments, the terms “sample”, “test sample” and “specimen” are used interchangeably in the present specification and claims and are used in its broadest sense. They are meant to include both biological and environmental samples and may include an exemplar of synthetic origin. This term refers to any media that may contain the at least one microorganism, e.g., a pathogen and may include fluid, cell and/or tissue samples. In some embodiments herein, the biological sample is a fluid sample. Fluid sample include, but are not limited to, saliva, mucosa, feces, serum, urine, blood, plasma, cerebral spinal fluid (CSF), milk, bronchoalveolar lavage (BAL) fluid, rinse fluid obtained from wash of body cavities, phlegm, pus. Still further, biological samples including samples taken from various body regions (nose, throat, vagina, ear, eye, skin, sores), food products (both solids and fluids) and swabs taken from medicinal instruments, apparatus, materials), samples from various surfaces [hospitals, elderly homes, food manufacturing facilities, slaughterhouses, pharmaceutical equipment (catheters etc), food preparation or packaging products), solutions and buffers], sewage etc. In some embodiments, the disclosed microorganism or pathogen-detecting methods may use any sample, for example, such sample may be a biological sample or an environmental sample. More specifically, biological samples may be provided from animal, including human, fluid, solid (e.g., stool) or tissue, as well as liquid and solid food and feed products, food designed for human consumption, a sample including food designed for animal consumption, food matrices and ingredients such as dairy items, vegetables, meat and meat by-products, waste and sewage. In some embodiments, biological samples may include saliva, mucosa (nasal or oral swab samples), feces, serum, blood, urine, anterior nares specimen collected by a healthcare professional or by onsite or home self-collection specimens throat swab. Biological samples and specimens may be obtained from human as well as from all of the various families of domestic animals, as well as feral or wild animals, including, but not limited to, such animals as ungulates, bear, birds, fish, lagamorphs, rodents, etc.

Still further, environmental samples include environmental material such as surface matter, earth, soil, water, air and industrial samples, as well as samples obtained from food and dairy processing instruments, apparatus, equipment, utensils, disposable and non-disposable items. These examples are not to be construed as limiting the sample types applicable to the present disclosure. The sample may be any media, specifically, a liquid media that may contain the target nucleic acid molecules or sequences. Typically, substances, surfaces and samples or specimens that are a priori not liquid may be contacted with a liquid media which is used and tested by the methods disclosed herein.

In some embodiments, the methods of the present disclosure may be applicable for detecting at least one microorganism, specifically, pathogen in food or food products and beverages. More specifically, by the term “food”, it is referred to any substance consumed, usually of plant or animal origin. Some non limiting examples of animals used for feeding are cows, pigs, poultry, etc. The term food also comprises products derived from animals, such as, but not limited to, milk and food products derived from milk, eggs, meat, etc. A drink or beverage is a liquid which is specifically prepared for human consumption. Non limiting examples of drinks include, but are not limited to water, milk, alcoholic and non-alcoholic beverages, soft drinks, fruit extracts, etc.

A further aspect of the present disclosure relates to a method of determining the genotype or the genetic profiling of at least one nucleic acid molecule of at least one organism, or of at least one infectious entity. In some embodiments, the profiling and/or genotyping is performed in at least one loci of interest, for example, at one or more polymorphic loci of interest. More specifically, the method comprising the step of performing molecular inversion probe-based targeted sequencing in at least one test sample comprising the at least one nucleic acid molecule. More specifically, the molecular inversion probe-based targeted sequencing method used herein comprising the step of:

In one step (a), contacting at least one MIP with at least one target nucleic acid sequence comprising the one or more polymorphic loci of interest, and incubating for a hybridization time of one to three and a half hours. In more specific embodiments, the MIP used in the disclosed methods may comprise: (i) a first region comprising a first sequence complementary to a first target region in the target nucleic acid sequence, and (ii) a second region comprising a second sequence complementary to a second target region in the target nucleic acid sequence. The first hybridization step results in MIP/s hybridized to the first and second target regions of the target nucleic acid sequence, that comprises the one or more polymorphic loci of interest.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. In some embodiments, the synthesized sequence is further ligated to obtain cyclized product/s in the reaction mixture. The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

The disclosed methods thus concern genotyping of a nucleic acid sequence. The term “genotyping” as herein defined refers to the identification of the nucleic acid sequence at specific loci in the DNA of an individual. As used herein, the terms “DNA profile,” “genetic fingerprint,” and “genotypic profile” are used interchangeably herein to refer to the allelic variations in a collection of polymorphic loci, such as a tandem repeat, a single nucleotide polymorphism (SNP), etc. A DNA profile is useful in forensics for identifying an individual based on a nucleic acid sample.

In some embodiments, the molecular inversion probe-based targeted sequencing method is performed in the disclosed genotyping method as defined by the present disclosure.

More specifically, in some embodiments, the hybridization time of the MIP-based targeted sequencing method used by the disclosed genotyping methods is less than three and a half hours.

In yet some further embodiments, the hybridization time of the MIP-based targeted sequencing method used by the disclosed genotyping methods is one to three hours.

Still further, in some embodiments, the hybridization time of the MIP-based targeted sequencing method used by the disclosed genotyping methods is one to two and a half hours.

Still further, in some embodiments, the step of enzymatic digestion of all linear MIPs and/or nucleic acid molecules that may be present in the reaction mixture obtained in step (b) of the MIP-based targeted sequencing method used by the disclosed genotyping methods, may last for about 15 to 30 minutes.

In some embodiments, the entire process that includes steps (a) to (c) of the of the MIP-based targeted sequencing method used by the disclosed genotyping methods is performed within less than 200 minutes. In some embodiments, the hybridization time is 153 minutes, the polymerization time is 10 minutes, and the digestion time is 30 minutes or 15 minutes, thereby, all three steps may be performed within 193 to 178 minutes. In some embodiments 193 or 178 minutes. Still further in some embodiments the hybridization time is 135 minutes, the polymerization time is 10 minutes, and the digestion time is 30 minutes or 15 minutes, thereby, all three steps may be performed within 175 to 160 minutes. In some embodiments 175 or 160 minutes.

In some embodiments, the hybridization time is 120 minutes, the polymerization time is 10 minutes, and the digestion time is 30 minutes or 15 minutes, thereby, all three steps may be performed within 160 to 145 minutes. In some embodiments 145 or 160 minutes. Still further, in some embodiments, the hybridization time is 103 minutes, the polymerization time is 10 minutes, and the digestion time is 30 minutes or 15 minutes, thereby, all three steps may be performed within 143 to 138 minutes. In some embodiments 143 or 138 minutes.

In some embodiments, the MIP-based targeted sequencing method used by the disclosed genotyping methods may use at least one MIP, specifically, a plurality of MIPs corresponding or targeted at, or specific for to a plurality of different target regions.

In yet some further embodiments, the MIP-based targeted sequencing method used by the disclosed genotyping methods may further comprise sequencing a plurality of synthesized sequences obtained in step (d) and identifying variants of interest.

Still further, in some embodiments, the MIP-based targeted sequencing method used by the disclosed genotyping methods may further comprise applying machine learning algorithm on the identified variants or a subgroup thereof, for calculating sensitivity, specificity and precision thereof. In some embodiments, the subgroup of variants comprises variants having VAF below threshold.

It should be noted that the at least one MIP used by the MIP-based targeted sequencing method used by the disclosed genotyping methods, may be a double strand probe. However, it should be appreciated that also single strand MIPs may be applicable in the disclosed methods.

It should be appreciated that in some embodiments the target nucleic acid sequence used in the genotyping methods, may be any genomic nucleic acid sequence. In yet some further embodiments, the target sequence may be transcriptomic nucleic acid sequence, thereby providing information with respect to the transcriptome and/or the exome of an organism.

In some embodiments, the target nucleic acid sequence is a nucleic acid sequence associated with, or comprising, at least one of: genetic and/or epigenetic variation/s, pathologic disorder/s, pathogenic entity, microorganism/s and GC-rich regions.

In more specific embodiments, genetic variations comprise at least one of: single nucleotide variant (SNVs) and/or single-nucleotide polymorphisms (SNPs), insertions and/or deletions, (indels), inversions, copy number variations (CNV), structural variations, alternative splicing, loss of heterozygosity (LOH), gene fusions, translocations, duplications and variable number of tandem repeats.

Still further, in some embodiments, the target nucleic acid sequence analyzed by the disclosed genotyping methods is associated with at least one congenital, spontaneous, or acquired pathologic disorder or condition. In yet some further embodiments, the pathologic disorder may be at least one of: a proliferative disorder, neoplastic disorder, a metabolic condition, mental disorders, an inflammatory disorder, an infectious disease caused by a pathogen, an autoimmune disease, a cardiovascular disease, a neurodegenerative disorder, fetal genetic condition and an age-related condition. Still further, pathologic disorders encompassed by the present disclosure further include infections and parasitic diseases, endocrine, nutritional diseases, immunity disorders, diseases of blood and blood forming organs, mental disorders, diseases of nervous system and sense organs, diseases of the circulatory system, diseases of the respiratory system, diseases of the digestive system, diseases of genitourinary system, complications of pregnancy, childbirth and the puerperium, diseases of the skin and subcutaneous tissue, diseases of musculoskeletal system and connective tissue and congenital anomalies.

In some embodiments, the genotyped organism is at least one organism of the biological kingdom Animalia, at least one organism of the biological kingdom Plantae, the biological kingdom Bacteria, the biological kingdom Archaea, the biological kingdom Protozoa, the biological kingdom Chromista and the biological kingdom Fungi.

Thus, the organism genotyped or genetically profiled by the disclosed methods may be any organism and/r any subject of any of the following biological kingdoms: Bacteria, Archaea, Protozoa, Chromista, Plantae, Fungi and Animalia.

More specifically, it should be understood that an organism of the Archaea kingdom in accordance with the present disclosure constitute a domain of single-celled organisms. These microorganisms lack cell nuclei and are therefore prokaryotes. Archaea are a major part of Earth's life. They are part of the microbiota of all organisms. In the human microbiome, they are important in the gut, mouth, and on the skin.

It should be understood that an organism of the Protozoa kingdom (singular protozoon or protozoan, plural protozoa or protozoans) in accordance with the present disclosure Protozoa is an informal term for a group of single-celled eukaryotes, either free-living or parasitic, that feed on organic matter such as other microorganisms or organic tissues and debris. The major groups of Protozoa includes but are not limited to: Flagellates, or Mastigophora (motile cells equipped with whiplike organelles of locomotion, e.g., Giardia lamblia); Amoebae or Sarcodina (cells that move by extending pseudopodia or lamellipodia, e.g., Entamoeba histolytica); Sporozoa, or Apicomplexa or Sporozoans (parasitic, spore-producing cells, whose adult form lacks organs of motility, e.g., Plasmodium knowlesi); Apicomplexa (now in Alveolata); Microsporidia (now in Fungi); Ascetosporea (now in Rhizaria); Myxosporidia (now in Cnidaria); Ciliates, or Ciliophora (cells equipped with large numbers of cilia used for movement and feeding, e.g. Balantidium coli).

Chromista is a biological kingdom consisting of single-celled and multicellular eukaryotic species that share similar features in their photosynthetic organelles (plastids). It includes all protists whose plastids contain chlorophyll c, such as some algae, diatoms, oomycetes, and protozoans. It is probably a polyphyletic group whose members independently arose as a separate evolutionary group from the common ancestor of all eukaryotes. As it is assumed the last common ancestor already possessed chloroplasts of red algal origin, the non-photosynthetic forms evolved from ancestors able to perform photosynthesis. Their plastids are surrounded by four membranes and are believed to have been acquired from some red algae. Chromista has been originally described as consisting of three different groups: Heterokonts or stramenopiles (brown algae, diatoms, water moulds, etc); aptophytes; and Cryptomonads.

It should be understood that an organism of the fungus kingdom is any member of the group of eukaryotic organisms that includes microorganisms such as yeasts and molds, as well as the more familiar mushrooms. The major phyla (sometimes called divisions) of fungi have been classified mainly on the basis of characteristics of their sexual reproductive structures. As of 2019, nine major lineages have been identified: Opisthosporidia, Chytridiomycota, Neocallimastigomycota, Blastocladiomycota, Zoopagomycota, Mucoromycota, Glomeromycota, Ascomycota and Basidiomycota.

It should be appreciated that organisms of the biological kingdom Animalia, or of the biological kingdom Plantae applicable in the present aspect are any of the organisms as defined in connection with other aspects of the present disclosure. Still further any bacterial organism and or any infectious entity (e.g., viruses, bacteriophages, or any transducing entity) disclosed by the present disclosure are also applicable in the present aspect.

The genotyping and genetic profiling methods disclosed by the reset disclosure can be useful in various applications, to name but few, such applications may include agriculture, health, parental testing, epidemiology, and forensic applications.

More specifically, in some embodiments, the disclosed genotyping and genetic profiling methods may be applied in Agricultural genomics, or agrigenomics (the application of genomics in agriculture). In some non-limiting embodiments, the methods disclosed herein may be applied in seed selection, livestock improvements. In some non-limiting examples, the methods disclosed herein identify genetic markers linked to desirable traits, informing cultivation and breeding decisions. In some other non-limiting examples, the methods disclosed herein may be useful to improve plant and animal selection, nutrition, health surveillance, traceability, and veterinary diagnostics systems. In some non-limiting examples, the methods disclosed herein may be applied in developing varieties of plant crops with, for example, desirable traits such as drought tolerance, disease resistance, and higher yield. The methods disclosed herein may be applied in agrigenomics for identifying and propagating genetic variants that confer beneficial agronomic traits, in complex environments, acquiring the ability to cope with elements in their environment such as predators, soil conditions, and climate. Examples of phenotypic traits of agriculture value include but not limited to yield and growth, disease resistance, abiotic stress adaptation, reproduction, nutrition/end-use quality, sustainability, etc.

The genotyping and genetic profiling methods disclosed herein may be applied in providing valuable information about the biological status of important resources like fisheries, crop and livestock health, and food safety and authenticity. The methods may be used to identify organisms present within various environments in order to understand ecosystem diversity. Species contribute DNA to their environment, which can be easily recovered and is often referred to as environmental DNA (eDNA), that may serve as a means of differentiating species based on a unique genetic fingerprint. In this way, eDNA is used to determine the repertoire of organisms present in any setting from seawater to soil and food. This and other emerging applications of genomics are shaping best practices for resource monitoring and management related to agriculture and may be use by the disclosed methods.

In some other embodiments, the disclosed genotyping and genetic profiling methods may be utilized by animal breeders. As used herein, the term “breeder animal” refers to a non-human animal (e.g., domestic animals as mammals, specifically horse, sheep, cows, dogs, etc. fish, and avian animals) used for breeding. Accordingly, a breeder animal may be one that is used for breeding using conventional means, such as, e.g., mating a male breeder animal with a female breeder animal. Alternatively, a breeder animal may be one that is used as a donor of genetic material (e.g., sperm, egg, or mitochondria of the breeder animal) for the purpose of producing an offspring animal having one or more predetermined traits in the absence of physical mating with another breeder animal. In cases where an offspring animal is produced without requiring mating between two breeder animals, the genetic source material may be obtained and used from a single breeder animal or in combination with genetic material from one or more additional breeder animals. Additionally, a breeder animal may be a living animal or a deceased animal. In the case of a deceased animal, genetic material is obtained from the animal antemortem and cryopreserved for later use in producing an offspring animal having one or more predetermined traits.

Still further, in some aspects thereof, the disclosed genotyping and genetic profiling methods may be applicable in forensic applications. More specifically, the use of a subset of markers in a human genome has been utilized to determine an individual's personal identity, or DNA fingerprint or profile. These markers include locations or loci of short tandem repeated sequences (STRs) and intermediate tandem repeated sequences (ITRs) which in combination are useful in identifying one individual from another on a genetic level. Accordingly, STR markers are frequently used in the fields of forensic analysis, paternity determination and detection of genetic diseases and cancers.

Thus, the genotyping and genetic profiling methods disclosed herein may be applicable for DNA profiling which may use in some non-limiting examples, selected biological markers for determining the identity of a DNA sample. For example, the most common analysis for determining a DNA profile is to determine the profile for a number of short tandem repeated (STRs) sequences found in an organism's genome. Species identification is one of most important components of forensic practice. For example, in some cases of poaching and trading of endangered species, it has been used to provide important information and assist in police investigations. In the food industry, identification of the species present in meat products can be achieved, and in archeology, human remains can be distinguished from non-human remains. Still further, a DNA profile is useful in forensics for identifying an individual based on a nucleic acid sample. DNA profile as used herein may also be used for other applications, such as diagnosis and prognosis of diseases including cancer, cancer biomarker identification, inheritance analysis, genetic diversity analysis, genetic anomaly identification, quantification of minority populations, databanking, forensics, criminal case work, paternity, personal identification, etc.

Further, the methods disclosed herein may apply to any organism, for example humans, non-human primates, animals, plants, viruses, bacteria, fungi and the like. As such, the present methods are not, only useful for DNA profiling (e.g., forensics, paternity, individual identification, etc.) and humans as a target genome, but could also be used for other targets such as cancer and disease markers, genetic anomaly markers and/or when the target genome is not human based.

Still further aspects of the present disclosure concerns genotyping and genetic profiling methods that may be applicable in microbiome analysis which allows one to identify and quantify (relatively) the microbial community in a given set of samples.

Still further, in some embodiments, the genotyping and genetic profiling methods of the present disclosure, may be used for tumor analysis. More specifically, tumor biopsies are often a mixture of health and tumor cells. Targeted PCR allows deep sequencing of SNPs and loci with close to no background sequences. It may be used for copy number and loss of heterozygosity analysis on tumor DNA. Said tumor DNA may be present in many different body fluids or tissues of tumor patients. It may be used for detection of tumor recurrence, and/or tumor screening.

In yet some further aspects thereof, the genotyping and genetic profiling methods of the present disclosure may be useful for diagnosis of fetal genetic abnormalities. In such case, the starting sample may be obtained from maternal tissue (e.g., blood, plasma) or may contain fetal samples (present in amniotic fluid). The methods described in the present disclosure apply techniques for allowing detection of small, but statistically significant, differences in polynucleotide copy number. The targets for the assays and MIP probes described herein can be any genetic target associated with fetal genetic abnormalities, including aneuploidy as well as other genetic variations, such as mutations, insertions, additions, deletions, translocation, point mutation, trinucleotide repeat disorders and/or single nucleotide polymorphisms (SNPs), as well as control targets not associated with fetal genetic abnormalities. Still further, in some embodiments, the methods and compositions described herein can enable detection of extra or missing chromosomes, particularly those typically associated with birth defects or miscarriage. For example, the methods and compositions described herein may enable detection of autosomal trisomies (e.g., Trisomy 13, 15, 16, 18, 21, or 22). In other cases, the trisomy that is detected is a liveborn trisomy that may indicate that an infant will be born with birth defects (e.g., Trisomy 13 (Patau Syndrome), Trisomy 18 (Edwards Syndrome), and Trisomy 21 (Down Syndrome)). The abnormality may also be of a sex chromosome (e.g., XXY (Klinefelter's Syndrome), XYY (Jacobs Syndrome), or XXX (Trisomy X). In some embodiments, the genetic target may be in any chromosome for example, 13, 18, 21, X or Y. Still further, to name but few, additional fetal conditions that can be determined based on the methods and systems herein include monosomy of one or more chromosomes (X chromosome monosomy, also known as Turner's syndrome), trisomy of one or more chromosomes (13, 18, 21, and X), tetrasomy and pentasomy of one or more chromosomes (which in humans is most commonly observed in the sex chromosomes, e.g. XXXX, XXYY, XXXY, XYYY, XXXXX, XXXXY, XXXYY, XYYYY and XXYYY), monoploidy, triploidy (three of every chromosome, e.g. 69 chromosomes in humans), tetraploidy (four of every chromosome, e.g. 92 chromosomes in humans), pentaploidy and multiploidy.

In some cases, the genetic target comprises more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 175, 200, 225, 250, 300, 350, 400, 450, 500, 1,000, 5,000, 10,000, 20,000, 30,000, 40,000, 50,000, 60,000, 70,000, 80,000, 90,000 or 100,000 sites on a specific chromosome. In some cases, the genetic target comprises targets on more than 1, 2, 3, 4, 5, 6, 7, 8, 9, 10, 11, 12, 13, 14, 15, 16, 17, 18, 19, 20, 21, or 22 different chromosomes. In some cases, the genetic target comprises targets on less than 2, 3, 4, 5, 6, 7, 8, 9, 10, 11, 12, 13, 14, 15, 16, 17, 18, 19, 20, 21, 22, or 23 chromosomes. In some cases, the genetic target comprises a gene that is known to be mutated in an inherited genetic disorder, including autosomal dominant and recessive disorders, and sex-linked dominant and recessive disorders. Non-limiting examples include genetic mutations that give rise to autoimmune diseases, neurodegenerative diseases, cancers, and metabolic disorders. In some embodiments, the method detects the presence of a genetic target associated with a genetic abnormality (such as trisomy), by comparing it in reference to a genetic target not associated with a genetic abnormality (such as a gene located on a normal diploid chromosome).

Still further, the disclosed genotyping and genetic profiling methods disclosed herein may be used for standard paternity and identity testing of relatives or ancestors, in human, animals, plants or other creatures. It may be used for rapid genotyping and copy number analysis (CN), on any kind of material, e.g., amniotic fluid and CVS, sperm, product of conception (POC). It may be used for single cell analysis, such as genotyping on samples biopsied from embryos. It may be used for rapid embryo analysis (within less than one, one, or two days of biopsy).

In some embodiments, the methods described herein may be used to identify SNPs, copy number, nucleotide methylation, mRNA levels, other types of RNA expression levels, other genetic and/or epigenetic features. The methods described herein may be used along with next-generation sequencing; it may be used with other downstream methods such as microarrays, counting by digital PCR, real-time PCR, Mass-spectrometry analysis etc.

A further aspect of the present disclosure relates to a method for identifying low variant allele frequency (VAF) mutations in a target nucleic acid molecule by performing molecular inversion probe-based targeted sequencing in said nucleic acid molecule. More specifically, the method comprising the step of:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence. The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the reaction mixture.

The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

In some embodiments, the molecular inversion probe-based targeted sequencing method is performed in the disclosed low VAF mutations detecting method as defined by the present disclosure.

More specifically, in some embodiments, the hybridization time is less than three and a half hours. In yet some further embodiments, the hybridization time is one to three hours.

Still further, in some embodiments, the hybridization time is one to two and a half hours. Still further, in some embodiments, the step of enzymatic digestion of all linear MIPs and/or nucleic acid molecules that may be present in the reaction mixture obtained in step (b), may last for about 15 to 30 minutes. In some embodiments, the entire process that includes steps (a) to (c) of the disclosed methods is performed within less than 200 minutes. In some embodiments, the hybridization time is 153 minutes, the polymerization time is 10 minutes, and the digestion time is 30 minutes or 15 minutes, thereby, all three steps may be performed within 193 to 178 minutes. In some embodiments, within 193 or 178 minutes. Still further in some embodiments the hybridization time is 135 minutes, the polymerization time is 10 minutes, and the digestion time is 30 minutes or 15 minutes, thereby, all three steps may be performed within 175 to 160 minutes. In some embodiments, within 175 or 160 minutes. In some embodiments, the hybridization time is 120 minutes, the polymerization time is 10 minutes, and the digestion time is 30 minutes or 15 minutes, thereby, all three steps may be performed within 160 to 145 minutes. In some embodiments, within 160 or 145 minutes. Still further, in some embodiments, the hybridization time is 103 minutes, the polymerization time is 10 minutes, and the digestion time is 30 minutes or 15 minutes, thereby, all three steps may be performed within 143 to 138 minutes. In some embodiments, within 143 or 138 minutes. In some embodiments the disclosed methods may use at least one MIP, specifically, a plurality of MIPs corresponding or targeted at, or specific for to a plurality of different target regions.

In yet some further embodiments, the disclosed method further comprise sequencing a plurality of synthesized sequences obtained in step (d) and identifying variants of interest.

Still further, in some embodiments, the disclosed method may further comprise applying machine learning algorithm on the identified variants or a subgroup thereof, for calculating sensitivity, specificity and precision thereof.

In some embodiments, the subgroup of variants comprises variants having VAF below threshold.

It should be noted that the at least one MIP used by the disclosed method may be a double strand probe. However, it should be appreciated that also single strand MIPs may be applicable in the disclosed methods.

A further aspect of the present disclosure relates to a method for performing molecular inversion probe-based targeted sequencing in at least one target nucleic acid sequence comprising at least one GC-rich region, the method comprising the step of:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the polymerization and/or ligation reaction mixture.

The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

In some embodiments, the molecular inversion probe-based targeted sequencing method is performed in the disclosed GC-rich region detecting method as defined by the present disclosure.

More specifically, in some embodiments, the hybridization time is less than three and a half hours.

In yet some further embodiments, the hybridization time is one to three hours.

Still further, in some embodiments, the hybridization time is one to two and a half hours. Still further, in some embodiments, the step of enzymatic digestion of all linear MIPs and/or nucleic acid molecules that may be present in the reaction mixture obtained in step (b), may last for about 15 to 30 minutes. In some embodiments, the entire process that includes steps (a) to (c) of the disclosed methods is performed within less than 200 minutes. In some embodiments, the hybridization time is 153 minutes, the polymerization time is 10 minutes, and the digestion time is 30 minutes or 15 minutes, thereby, all three steps may be performed within 193 to 178 minutes. In some embodiments, within 193 or 178 minutes. Still further in some embodiments the hybridization time is 135 minutes, the polymerization time is 10 minutes, and the digestion time is 30 minutes or 15 minutes, thereby, all three steps may be performed within 175 to 160 minutes. In some embodiments, within 175 or 160 minutes. In some embodiments, the hybridization time is 120 minutes, the polymerization time is 10 minutes, and the digestion time is 30 minutes or 15 minutes, thereby, all three steps may be performed within 160 to 145 minutes. In some embodiments, within 160 or 145 minutes. Still further, in some embodiments, the hybridization time is 103 minutes, the polymerization time is 10 minutes, and the digestion time is 30 minutes or 15 minutes, thereby, all three steps may be performed within 143 to 138 minutes. In some embodiments, within 143 or 138 minutes. In some embodiments the disclosed methods may use at least one MIP, specifically, a plurality of MIPs corresponding or targeted at, or specific for to a plurality of different target regions.

In yet some further embodiments, the disclosed method further comprise sequencing a plurality of synthesized sequences obtained in step (d) and identifying variants of interest.

Still further, in some embodiments, the disclosed method may further comprise applying machine learning algorithm on the identified variants or a subgroup thereof, for calculating sensitivity, specificity and precision thereof.

In some embodiments, the subgroup of variants comprises variants having VAF below threshold.

It should be noted that the at least one MIP used by the disclosed method may be a double strand probe. However, it should be appreciated that also single strand MIPs may be applicable in the disclosed methods.

A further aspect provide by the present disclosure relates to a method for improving the performance of molecular inversion probe-based targeted sequencing in at least one of: uniformity, on-target reads and GC-rich regions coverage, by shortening the incubation time of at least one of: (a) hybridization time of the at least one MIP with a target nucleic acid sequence to one to three and a half hours; (b) polymerization reaction to 1 to 20 minutes; and (c) enzymatic digestion for 10 to 45 minutes.

In some embodiments, the molecular inversion probe-based targeted sequencing method improved by the disclosed improving method as defined by the present disclosure.

More specifically, such improved method comprises the following steps:

One step (a), involves contacting at least one molecular inversion probe (MIP) with at least one target nucleic acid sequence, and incubating the MIP with the target sequence for a hybridization time of one to three and a half hours. In some embodiments, the MIP provided in the present method comprises: (i) a first region comprising a first sequence complementary to a first target region in the target nucleic acid sequence, and (ii) a second region comprising a second sequence complementary to a second target region in the target nucleic acid sequence, thereby obtaining a MIP hybridized to the first and second target regions of the target nucleic acid sequence.

The next step (b) involves subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP. It should be understood that the synthesized sequence is further ligated to obtain cyclized product/s in the reaction mixture.

The disclosed method may further comprise in some embodiments thereof, at least one additional step, specifically, at least one of steps (c) and (d). Thus, in some optional embodiments, the method may comprise a step of enzymatic digestion. More specifically, the next step (c) involves subjecting the reaction mixture obtained in step (b) to enzymatic digestion for 10 to 45 minutes, thereby digesting any linear MIP/s or linear nucleic acid molecule/s present in the reaction mixture. In yet some further embodiments, the disclosed methods may further comprise amplification step (d). Thus, in some embodiments, the next step (d) involves amplifying the synthesized sequence of the cyclized product/s.

In some embodiments, the hybridization time is less than three and a half hours.

In yet some further embodiments, the hybridization time is one to three hours.

Still further, in some embodiments, the hybridization time is one to two and a half hours. Still further, in some embodiments, the step of enzymatic digestion of all linear MIPs and/or nucleic acid molecules that may be present in the reaction mixture obtained in step (b), may last for about 15 to 30 minutes. In some embodiments, the entire process that includes steps (a) to (c) of the disclosed methods is performed within less than 200 minutes. In some embodiments, the hybridization time is 153 minutes, the polymerization time is 10 minutes, and the digestion time is 30 minutes or 15 minutes, thereby, all three steps may be performed within 193 to 178 minutes. In some embodiments, within 193 or 178 minutes. Still further in some embodiments the hybridization time is 135 minutes, the polymerization time is 10 minutes, and the digestion time is 30 minutes or 15 minutes, thereby, all three steps may be performed within 175 to 160 minutes. In some embodiments, within 175 or 160 minutes. In some embodiments, the hybridization time is 120 minutes, the polymerization time is 10 minutes, and the digestion time is 30 minutes or 15 minutes, thereby, all three steps may be performed within 160 to 145 minutes. In some embodiments, within 160 or 145 minutes. Still further, in some embodiments, the hybridization time is 103 minutes, the polymerization time is 10 minutes, and the digestion time is 30 minutes or 15 minutes, thereby, all three steps may be performed within 143 to 138 minutes. In some embodiments, within 143 or 138 minutes. In some embodiments the disclosed methods may use at least one MIP, specifically, a plurality of MIPs corresponding or targeted at, or specific for to a plurality of different target regions.

In yet some further embodiments, the disclosed method further comprise sequencing a plurality of synthesized sequences obtained in step (d) and identifying variants of interest.

Still further, in some embodiments, the disclosed method may further comprise applying machine learning algorithm on the identified variants or a subgroup thereof, for correcting enzymatic and chemical biases that naturally occur in library preparation. The said algorithm more accurately calculates VAF, and increases sensitivity, specificity and precision thereof.

In some embodiments, the subgroup of variants comprises variants having VAF below threshold.

It should be noted that the at least one MIP used by the disclosed method may be a double strand probe. However, it should be appreciated that also single strand MIPs may be applicable in the disclosed methods.

In some aspects thereof, the present disclosure further provides a kit adapted for performing the molecular inversion probe-based targeted sequencing of the present disclosure. In some particular embodiments, the kit may comprise hybridization mixture comprising hybridization buffer, for example, ampligase reaction buffer. In yet some further embodiments the polymerization reaction buffer may comprise at least one of Q5 High GC Enhancer, beta-nicotinamide adenine dinucleotide (NAD+), dNTPs, betaine, and an appropriate DNA polymerase, specifically, the Q5 high fidelity DNA polymerase.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to +10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understand to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

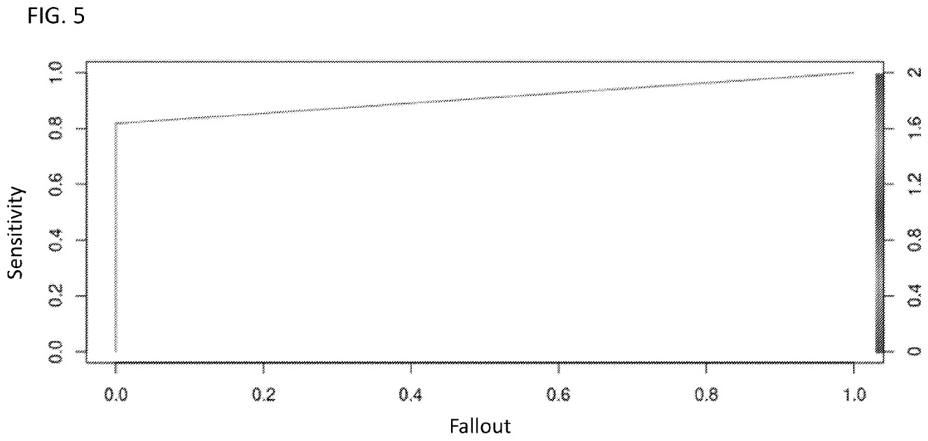

Biological Resources: DNA samples were obtained from donors considered healthy without known ARCH defining mutations in their clinical records. Per reaction a total DNA of 50-500 ng/ul was used. IP Targeted Sequencing probe design: Molecular inversion probes (MIP) capture probes were designed using MIPgen [2] to capture ARCH related targets (FIG. 5) (Shlush L. I. Blood. 2018; 131:496-504; Tuval A., Shlush L. I., Haematologica. 2019; 104:872-880) or a genotyping panel (FIG. 2). MIPs were either single strand MIPs (prepared as in [3]) or as oligo mix (LCsciences, prepared as in Shen et al., Genome Med., 5:50, 2013).

Multiplex MIP Capture protocol: 1 μl DNA template was added to a hybridization mix together with a MIP pool (final concentration of 0.05 pM per probe) in 1× Ampligase buffer (Epicentre). Mix was incubated in a thermal cycler at 98° C. for 3 minutes, followed by 85° C. for 30 minutes, 60° C. for 60 minutes and 56° C. for 1 or 2 overnight incubation periods. Product was mixed with dNTPs (15 pM), Betaine (375 mM), NAD+ (1 mM), additional Ampligase buffer (0.5×), Ampligase (total of 1.25 U) and Phusion HF (0.16 U). Mixture was incubated at 56° C. for 60 minutes followed by 72° C. for 20 minutes. Enzymatic digestion of linear probes was performed by adding Exonuclease I (4 U) and Exonuclease III (25 U). Mixture was incubated at 37° C. for 2 hours, followed by 80° C. for 20 minutes. Final product was amplified using iProof HF Master Mix (Biorad). Samples were pooled and concentrated, size-selected (190-370 bp) and sequenced using custom primers. In total, 4417 healthy individual DNA samples were processed and sequenced, twice each, as true technical duplicate using the above MIP protocol.

Improved MIP (iMIP) protocol: 1 μl DNA template was added to a hybridization mix together with a MIP pool (final concentration of 0.04 pM per probe) in 0.85× Ampligase buffer. Mix was incubated in a thermal cycler at 98° C. for 3 minutes, followed by 85° C. for 30 minutes, 60° C. for 60 minutes and 56° C. for 60 minutes (total of 153 minutes). Product was mixed with: dNTPs (14 pM), Betaine (375 mM), NAD+ (1 mM), additional Ampligase buffer (0.5×), Ampligase (total of 1.25 U) and Q5 High-Fidelity DNA Polymerase (0.4 U). Mixture was incubated at 56° C. for 5 minutes followed by 72° C. for 5 minutes. Enzymatic digestion of linear probes was performed by adding Exonuclease I (8 U) and Exonuclease III (50 U). Mixture was incubated at 37° C. for 10 minutes, followed by inactivation of the exonucleases in 80° C. for 20 minutes. Final product was amplified using NEBNext Ultra II Q5 Master Mix (New England Biolabs). Samples were pooled and concentrated using beads at 0.75× volumetric concentration and sequenced as abovementioned described.

To reduce the turnaround time, the following two alternative shorter iMIP hybridization programs were used:

a) Mix was incubated in a thermal cycler at 98° C. for 3 minutes, followed by 85° C. for 20 minutes, 61° C. for 40 minutes and 56° C. for 40 minutes (total of 103 minutes).

b) Mix was incubated in a thermal cycler at 98° C. for 3 minutes, followed by reducing temperature at Ramp temperature of −0.1° C.\sec between 98° C.-56° C., and 56° C. for 120 minutes (total of 135 minutes).

Furthermore, the exonuclease may be inactivated in 80° C., 90° C., or 95° C. for 5 minutes.

Amplicon sequencing for suspected variants detected in MIP protocol: Selected MIP probes were ordered as amplicon primers to enable target amplification using 2-step amplicon sequencing. After collecting all potential variants, the amplifying MIPs were sorted by the number of mutations in the cohort they will capture (highest first). MIPs were then converted to corresponding amplicons: to this end, the ligation arm was converted by “reverse complement”. 5′ tail addition and index primers were as previously described (Biezuner T., et al., Genome Res. 2016; 26:1588-1599). All selected amplicon primers were applied to all DNA samples in the experiment, generating a majority of sequencing data with no expected mutations at any sampled genomic region. This further allowed for per position true/false positive statistical validation. Selected primers were mixed in pools of ≤6 primer pairs/mix at a concentration of 2.5 uM per primer. 1st PCR reaction was performed by mixing NEBNext Ultra II Q5 Master Mix, lul DNA template, and primer mix (0.5 uM). PCR program: 98° C. activation for 30 seconds, followed by 5 steps of: denaturation at 98° C., annealing at 60° C. and extension at 65° C., then 25 steps of: Denaturation at 98° C., annealing and extension at 65° C. Final extension was at 65° C. for 5 minutes. Reaction was diluted 1:1000 and 2nd PCR (barcoding PCR) was at the same composition and protocol as the 1st PCR besides the reduction of the 2 step from 25 to 12 cycles. Reactions were pooled at equal volumes and purified by AMPure XP beads at 0.7× volumetric concentration, size-selected (265-400 bp) using Blue Pippin and sequenced in Novaseq6000 2×151 bp paired-end run.

Data preprocessing and variant calling: Paired-end 2×151 bp sequencing data were converted to FASTQ format. Reads were merged using BBmerge v38.62 with default parameters, followed by trimming of the ligation and extension arm using Cutadapt v2.10. Unique Molecular Identifiers (UMI) were trimmed and assigned to each read header. Processed reads were aligned using BWA-MEM (Li H. Aligning sequence reads, clone sequences and assembly contigs with BWA-MEM. 2013; arXiv doi: 26 May 2013, preprint: not peer reviewed) to a custom reference genome, comprised of the MIP ARCH panel sequences+150 bases extracted from broad HG19 [https://gatk.broadinstitute.org/hc/en-us/articles/360035890711-GRCh37-hg19-b37-humanG1Kv37-Human-Reference-Discrepancies #b37]. Aligned files were sorted, converted to BAM (SAMTools V1.9 (Li H. et al., Bioinformatics. 2009; 25:2078-2079, followed by Indel realignment using AddOrReplaceReadGroups (Picard tools) and later IndelRealigner (GATK v.3.7, McKenna A., et al., Genome Res. 2010; 20:1297-1303). Variant calling was done using mpileup for the single nucleotide variant (SNVs), and Varscan2 v2.3.9 (Koboldt D. C., et al., Genome Res. 2012; 22:568-576) and Platypus v0.8.1 (Rimmer A., et al., Nat. Genet. 2014; 46:912-918) for indels. Variants were annotated using ANNOVAR (Wang K., et al., Nucleic Acids Res. 2010; 38: e164).

Statistical analysis of SNVs for MIPs and amplicon: The depth for reference calls and all possible variants of all positions was retrieved from the mpileup files. Only positions with depth>100 were included. To estimate background error rate at each position first the total read depth was calculated across all samples (DEPTH_SUM) and the alternate supporting reads (ALT_READS_SUM). Next, the number of alternate reads in a sample (n) and the total depth for the sample in that position (N) were analyzed followed by the calculate of m=ALT_READS_SUM−n and M=DEPTH_SUM−N. For MIPs this was done separately on each technical duplicate. To test whether a specific VAF is significantly different from the background error rate the distribution of the variant was approximated using Poisson distribution and then poisson exact test was used on each variant estimation (stats R package) and corrected for multiple hypothesis testing with Benjamini Hochberg (BH) test per p-value to get a BH score.

Calculating expected number of duplicate and duplication ratio: To utilize the information from the large number of samples sequenced with the MIP panel (N=4417), and the fact they were all had technical duplicates, led to the addition another layer of data dealing with the duplicate's reproducibility. Accordingly, mplieup files of the technical duplicates were merged to define consensus positions that have depth >100 in both duplicates. Each variant was defined as singleton if identified in one of the technical duplicates or as a duplicate if found in both. Next mplieup files of all sample IDs were merged and the number of singleton (single_n) and duplicates (dup_n) in the entire dataset was calculated. The same counting was also performed only on variants with VAF 0.006 to define single_cutoff and dup_n_cutoff. The expected number duplicates for each variant was calculated

$$exp\_dups = \frac{single\_n {\_cutoff}^{2}}{total\_sample \_ids}$$

and the same for the duplicate ratio (dup_ratio)

$$dup\_ratio = \frac{dup\_n \_cutoff}{exp\_dups} .$$

Amplicon sequencing validation: In order to understand the MIP noise model, MIP sequencing was compared to amplicon sequencing. The targets for amplicon sequencing were chosen based on VAF true variants identified by the Poisson exact test. The focus was on variants known to play a role in ARCH where variants with BH1 and BH2<0.002 were selected to be validated by amplicon sequencing. To build the noise model of the amplicon sequencing approach this experiment was extended by targeting all samples in the experiment with all participating primers. This validation was performed in two iterations: the first iteration was composed of 84 DNA templates, and 48 amplicons covering 7930 bp. The second iteration was composed of 125 DNA templates, and 48 amplicons covering 7114 bp.

Calculating background error rates: For the calculation of background error rate, the mplieup files were filtered for variants with VAF<0.05 Depth >100. Background errors were calculated as the number of alternate reads over all sequenced bases in the same position across the entire panel. Error rates were evaluated for MIP amplicon and iMIP.

Refining low VAF detection in MIP sequencing: As the background noise of MIP was significantly higher than amplicon, ‘amplicon calling’ were used as true positives. True variants were defined in the amplicon sequencing based on the poisson exact test (p=0, depth >100 VAF>0.005), which identified N=42 true variants. SNVs in the MIP data were then called by calculating poisson exact test p values for both duplicates. The data was transformed to fit machine learning prediction algorithms. Next, various machine learning algorithms were applied, and it was decided to continue with SVM and the vanilladot Kernel (caret library R 4.0.4) to calculate sensitivity, specificity and precision of the SVM predictions (FIG. 5).

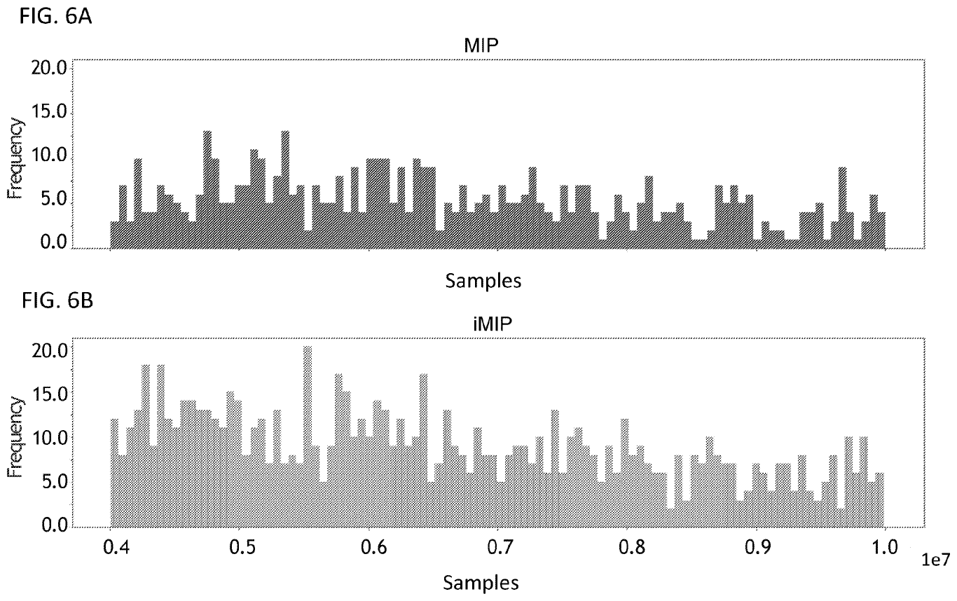

Comparing MIP and iMIP performance: To be able to compare the MIP and iMIP protocols samples that had similar depth distributions in the original FASTQ files were selected based on Kolomogorov Smirnov p value (FIGS. 6A and 6B, respectively), MIP N=535 and iMIP N=905 samples. To evaluate the number of MIPs that were covered sufficiently across samples the number of targets which received above 100 reads in at least one sample were compared, these MIPs were defined as working MIPs. Uniformity was calculated by the

$$\frac{\% MIPs with depth > ( 0.2 * mean depth )}{panel size} .$$

On-target rate was measured by the

$$\frac{\% Mapped reads}{total reads} .$$

Defining GC rich targets: The MIP target sequence was retrieved, and the GC content was evaluated using gc5Base table from UCSC table browser. GC rich regions were defined as regions with GC content >55%. From all working MIPs GC rich MIPs were identified and grouped by genes.

Genotyping panel: To test the ability of iMIP to capture large number of probes MIPgen was used to design a large panel of 8349 probes which capture SNPs. Such panel can be used for de-multiplexing human samples from pools of samples. Once it was discovered that a small subset of the MIPs of the present disclosure captured large proportion of reads, and that many MIP did not perform optimally, a set of 4409 was selected from the original panel and sequenced with it 104 samples with minimum depth of 10e6 reads.

### Example 1

### Improving MIP Noise Model

As detailed below, the MIP based targeted sequencing method disclosed herein exhibits improved performance. Using the MIP protocol, 4417 samples were sequenced in duplicates using the ARCH panel. This panel is composed of 707 MIP probes targeting 70134 genomic bases, of which, 616 probes were used for the analysis (‘working MIPs).

The current noise model used for low VAF calling after MIP targeted sequencing is generally based on a Poisson exact test and correction for multiple hypothesis. Furthermore, previous methods for error correction were applied for UMI deduplication to minimize noise; however, the UMI collapsing could not be used as the majority of read families in the present disclosure have a size of less than 5 reads per family/group (which is the standard cutoff for consensus sequence). The reason for the low number of families with more than 5 reads per family was the low total number of reads that was allocated for each sample in the disclosed study. As the aim was to detect low VAF variants in a cost-effective manner, intentionally lower coverage than needed for the use of UMIs, has been applied.

In order to develop new methods for error correction under the MIP targeted sequencing protocol without necessarily taking UMIs into account, the background error rate of amplicon and MIP sequencing were compared. Amplicon sequencing yielded significantly reduced error rate in all possible single nucleotide variants (SNV) alterations (FIG. 1A). A bimodal noise distribution in C>A was noticed in the MIP protocol in all MIP experiments ruling out the chance for a batch effect. This could be explained by DNA damage introduced during the library preparation process. The high background error rate produced by the MIP protocol suggests that the current state of the art statistical noise reduction tools for MIP might produce substantial false positive rates. Furthermore, the lower background error rate of the amplicon protocol suggests that the statistical noise detection could be improved by training a model on variants with higher probability of being true as they were validated by amplicon sequencing. Accordingly, true variants were defined using strict statistical cutoff on the amplicon sequencing data and 42 true variants were identified.

To evaluate the performance of the current state of the art statistical noise reduction algorithm, it was applied on the MIP data and compared to the true variants extracted from the amplicon sequencing. The outcome of this calculation yielded a specificity

$$( \frac{T N}{T N + F P} )$$

of was 99.74%, sensitivity

$$( \frac{T P}{T P + F N} )$$

of 80.95%, and precision

$$( \frac{T P}{T P + F P} )$$

of 10% (FIG. 1B). To improve the precision of the disclosed method machine learning algorithms that took into account only the parameters used in the past (VAF, Depth and Poisson exact test p values of the duplicates), were used. While this approach improved precision (50%) sensitivity was significantly lower (16.67%; p=0.004). Next, the hypothesis that adding information on the number of samples sequenced, duplicate ratio and other parameters extracted from the large dataset might improve the prediction model, has been tested. An SVM model was used, which yielded the following results: specificity of 99.98%, sensitivity of 81.81%, and significantly higher precision of 56.25% (p=1.4E-5; FIG. 1B). Altogether, as shown in FIG. 1B, the protocol developed herein significantly reduced the number of false positive variants.

### Example 2

### Refining the Biochemistry of the MIP Protocol to Improve Performance and to Reduce Noise

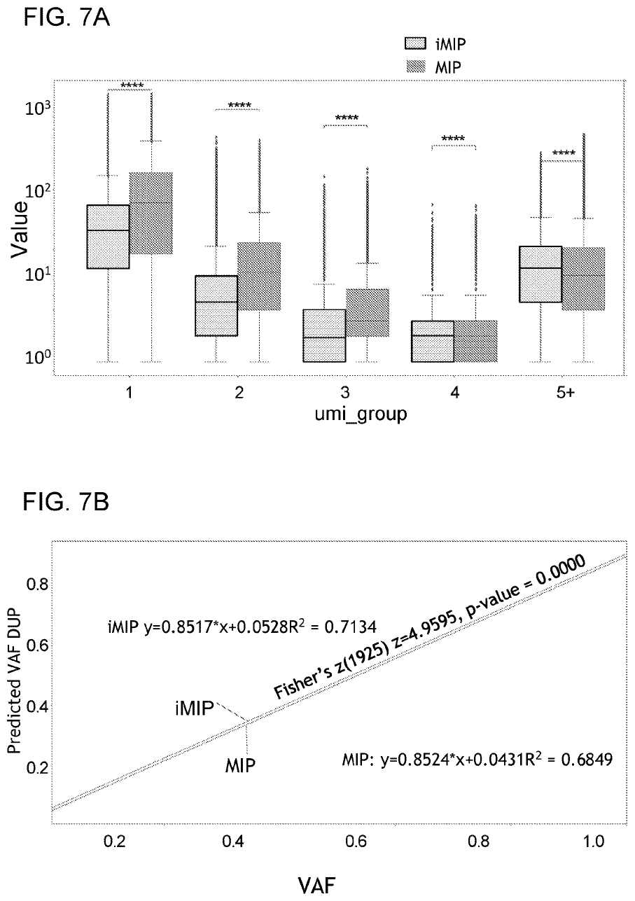

In addition to reducing the false positive rate of the MIP protocol, the MIP protocol steps were recalibrated, and the initial protocol's timing was reduced to under 4 hours (end to end). New 1569 samples were analyzed using the MIP ARCH panel mentioned above and the improved MIP protocol (iMIP). The results demonstrated significantly lower background error rate in the iMIP protocol versus the previous MIP protocol for all possible alterations, except for T>C (FIG. 2A). Furthermore, the iMIP protocol had a significant lower background error rate compared to amplicon sequencing in T>G and C>A transversions, while in other alterations amplicon sequencing was still superior (FIG. 2A). Of note the iMIP protocol had fewer small families (<5) and more large families (>5; FIG. 7A).

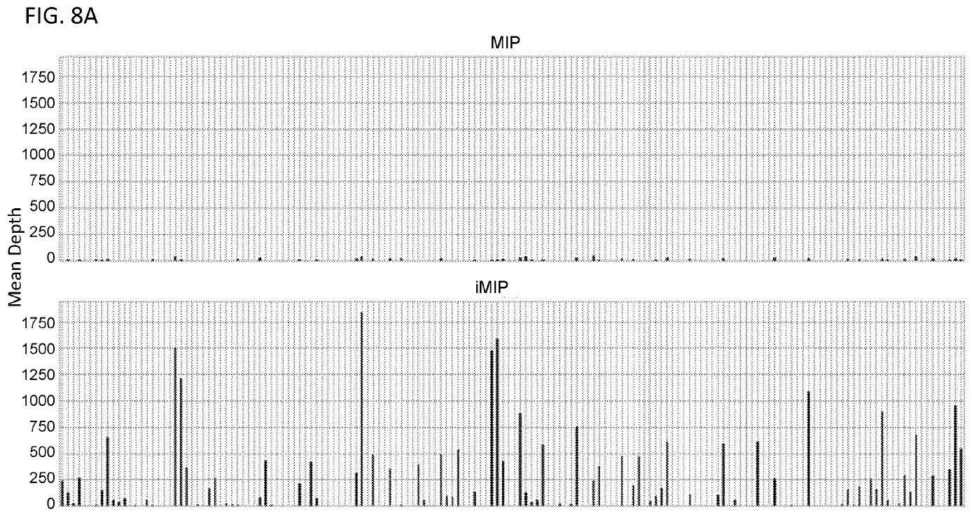

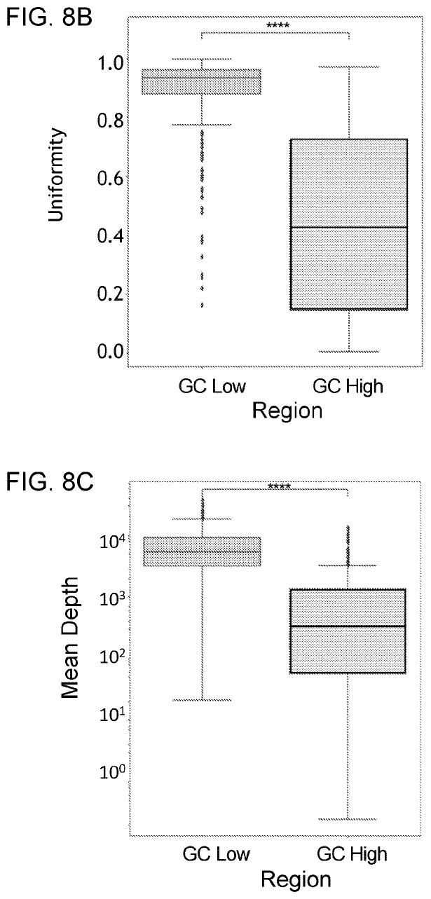

To study the effect of the iMIP protocol disclosed herein on the panel performance, the median number of MIPs that work was compared for both MIP and iMIP protocols and demonstrated a significant increase in the median MIPs that work in the iMIP protocol (609 versus 558 respectively p<0.00001; FIG. 2B). The iMIP protocol further demonstrated a significant improvement of in the panel uniformity (FIG. 2C) and the on-target rate (FIG. 2D). Of note the iMIP protocol had less small families (<5) and more large families (>5) (FIG. 7B). The next aim was to improve the uniformity and on-target rate specifically in the GC-rich regions, as it was reported in the past that MIP protocols perform poorly in such regions. Indeed, many of the MIPs that provided poor coverage in the MIP protocol, and which had better coverage in the iMIP protocol-exhibited high GC rich content (FIG. 8A). In the MIP protocol uniformity and mean depth were significantly lower in GC rich regions (FIGS. 8B and 8C, respectively). Furthermore, important GC rich regions, such as, the gene CEBPA and others barely had any coverage. To resolve these issues, the iMIP protocol disclosed herein, has been created. Indeed, this protocol provided significantly higher coverage across GC rich regions for all regions besides MIPs in the gene SETBP1 (FIG. 3A). Overall uniformity was also significantly higher in the iMIP protocol (FIG. 3B). Specifically in the GC rich region of CEBPA which is known to be a challenging region across various NGS technologies, the coverage by the iMIP protocol was significantly improved (FIG. 3C).

### Example 3

Performance of the iMIP Protocol on a Large Panel of 8349 Targets

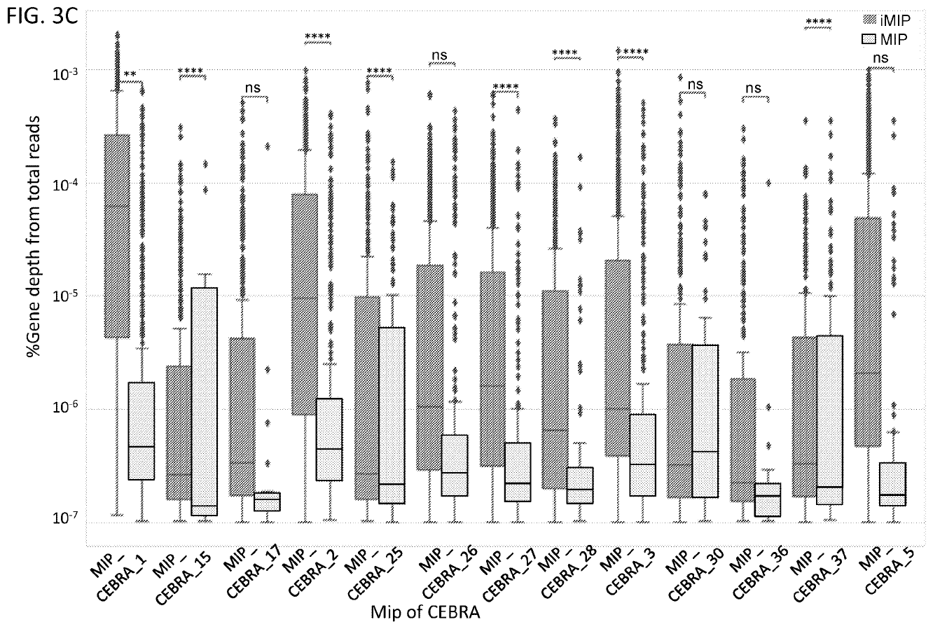

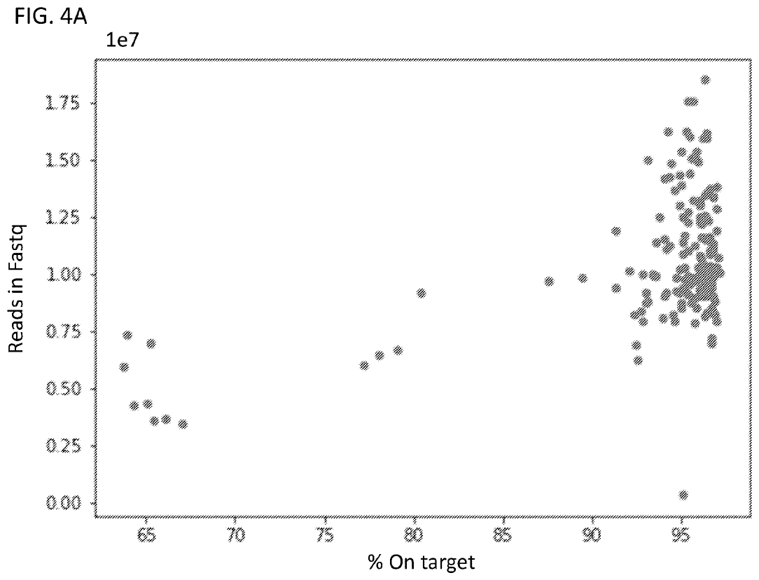

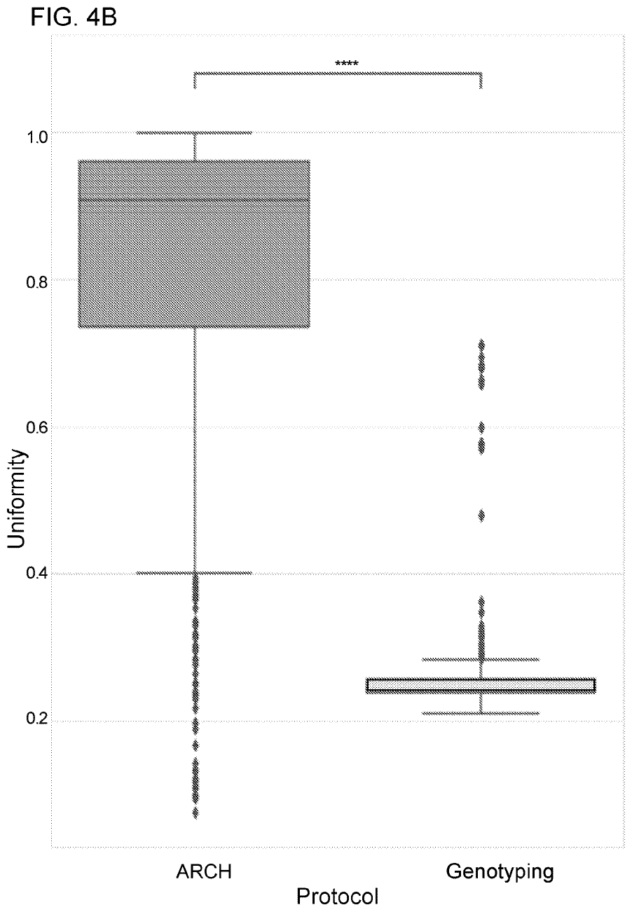

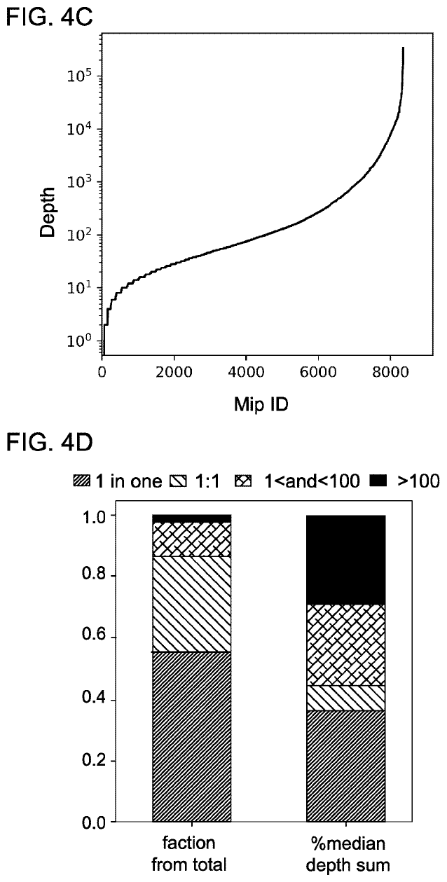

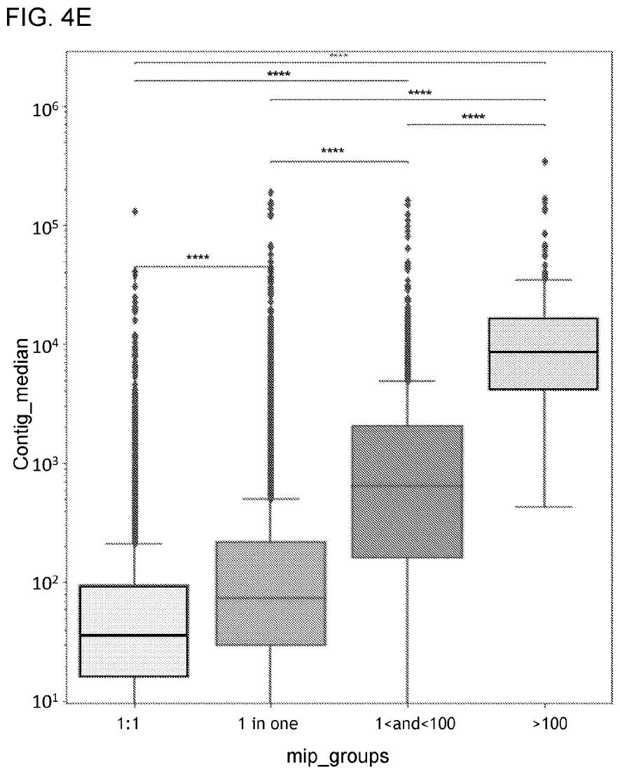

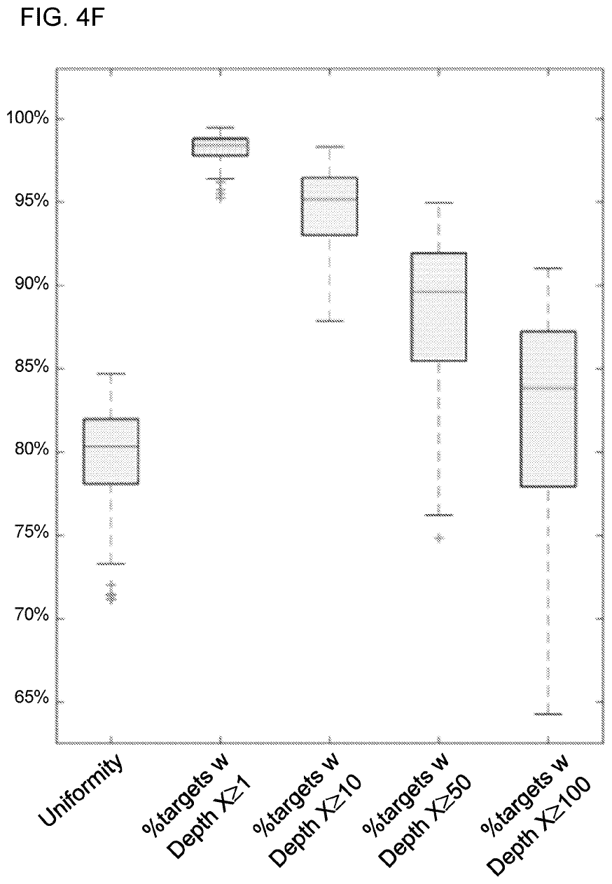

Next, to examine iMIP performance in larger MIP panels, the iMIP protocol was tested with a different genotyping panel containing 8349 MIPs. The results initially demonstrated that samples with more than one million reads in FASTQ had on average 95% of reads on target (FIG. 4A). However, compared to the uniformity of the latter panel to that of the smaller ARCH panel, the large panel resulted with a significantly lower uniformity (FIG. 4B). In order to better understand this low uniformity, the MIP properties of the mapped data were analyzed showing that a significant low number of MIPs took over large proportion the mapped reads (FIG. 4C), and also many MIPs did not work as good as others. By back tracing the origin of these MIPs, it has been found that some of these MIPs share higher copy number in their arms (FIG. 4D). Interestingly, although no copy number filter was provided upon ordering the MIP panel, there are two significant MIP arm copy number groups: <100 and >10000. These groups are significantly clustered and demonstrate the importance of this when designing panels. Analysis of the median depth of MIPs with different copy numbers showed a significant increase in coverage across MIPs with higher copy number (FIG. 4E). As the recommendations regarding arm copy number filtering are not clear, the uniformity across the different copy number groups was analyzed and it has been concluded that the best uniformity was achieved while choosing MIPs with copy number of one in at least one of the arms and the copy number in the other arm can be any number greater than one. To validate this hypothesis and to improve the performance of the tested genotyping panel, a reduced genotyping panel in that contained only MIPs with copy number of one in at least one of the arms. MIPs that demonstrated low coverage were removed from the reduced genotyping panel. Next, 104 samples with the reduced genotyping panel were sequenced and a median uniformity of 80.3% and median 50× coverage of 89.6% was achieved (FIG. 4F). Thus, the results demonstrate the ability of the iMIP protocol to target thousands of genomic targets.

### Example 4

Performance of Alternative Shorter and Cost-Effective iMIP Protocols

To further reduce costs and/or turnaround time, while maintaining and/or improving the uniformity and/or on-target rates of the disclosed iMIP, the inventors proceeded to modify certain parameters in the disclosed iMIP protocol (above under the “Improved MIP (iMIP) capture protocol”).

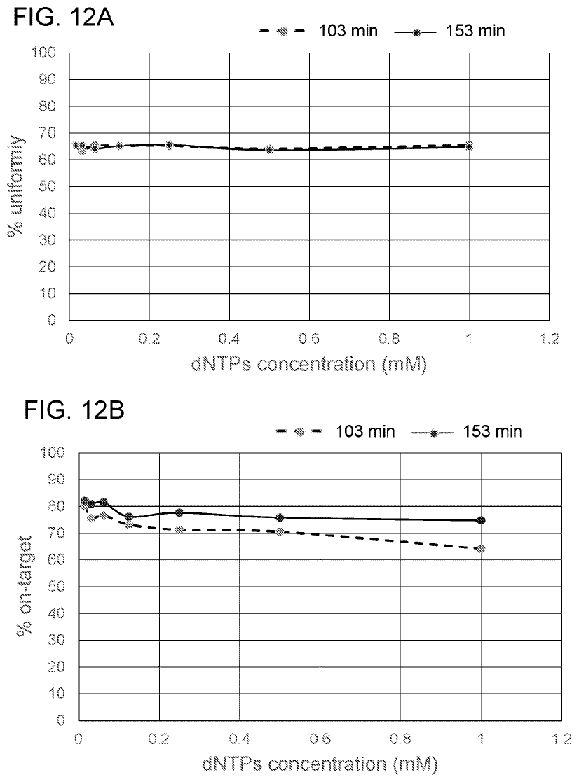

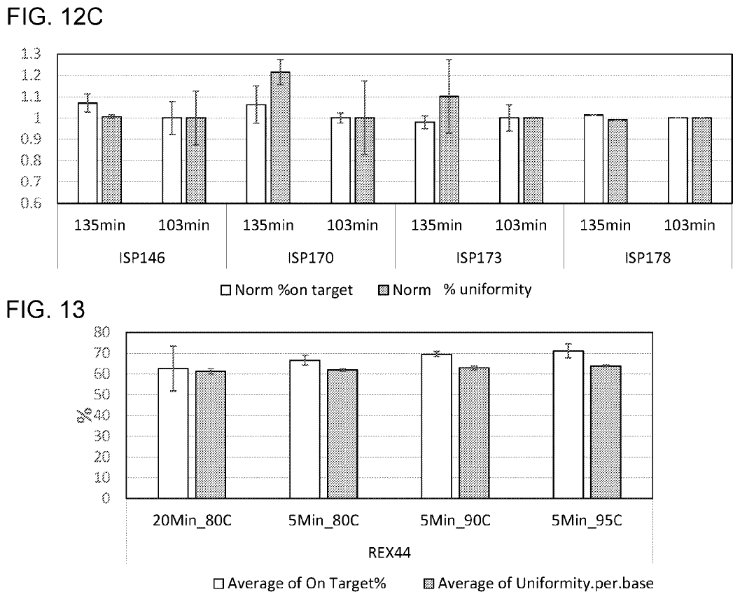

To that end, the inventors initially aimed to utilize shorter hybridization protocols in the attempt to reduce overall turnaround time. Accordingly, a comparison of the uniformity and on-target rates was performed between the iMIP hybridization protocol (153 minutes) to a shorter hybridization protocol (103 minutes), over various concentrations of dNTPs in the gap filling mix (FIGS. 12A and 12B). Substantially similar rate of uniformity (FIG. 12A) and target coverage of 100%>20× (data not shown) were obtained for both iMIP and the shorter protocol across all dNTPs concentrations. While the shorter hybridization protocol resulted in a moderate on-target rate reduction (FIG. 12B), it overall improved the turnaround time. To further improve the on-target rate while reducing the overall turnaround time of the iMIP protocol (153 minutes), another hybridization protocol with a gradual temperature decrease (135 minutes) was compared to the shorter hybridization protocol (103 minutes). Indeed, the hybridization protocol with the gradual temperature decrease (135 minutes) demonstrated substantially improved on-target rate with similar uniformity relative to the shorter protocol (FIG. 12C). The inventors were also able to save costs at this stage, by replacing the Ampligase reaction buffer in the hybridization step with the less expensive Q5 reaction buffer (supplied with the enzyme: Q5 High-Fidelity DNA Polymerase-NEB #B9027), without affecting the uniformity and/or on-target rates (data not shown).

Furthermore, the inventors aimed to further reduce the overall turnaround time by cutting the exonuclease inactivation incubation period in the iMIP protocol. The inventors found that inactivation of the exonuclease in 90° C. or 95° C. for 5 minutes instead of 80° C. for 20 minutes further reduces the overall turnaround time by 15 minutes, but still maintaining the average on-target and uniformity rates (FIG. 13).

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1-36. (canceled)

37. A molecular inversion probe-based targeted sequencing method, comprising the steps of:
a. contacting at least one molecular inversion probe (MIP) with at least one target nucleic acid sequence, and incubating for a hybridization time of one to three and a half hours, said MIP comprising:
(i) a first region comprising a first sequence complementary to a first target region in said target nucleic acid sequence, and
(ii) a second region comprising a second sequence complementary to a second target region in said target nucleic acid sequence; thereby obtaining a MIP hybridized to the first and second target regions of the target nucleic acid sequence; and
b. subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP, wherein the synthesized sequence is further ligated to obtain cyclized product/s in the polymerization and/or ligation reaction mixture; and optionally, at least one of:
c. subjecting the reaction mixture obtained in step (b) to enzymatic digestion for 10 to 45 minutes, thereby digesting linear MIP/s or nucleic acid molecule/s present in said reaction mixture; and
d. amplifying the synthesized sequence of said cyclized product/s.

38. The method of claim 37, wherein at least one of:
(i) the hybridization time is less than three and a half hours;
(ii) the hybridization time is one to three hours; and/or
(iii) the hybridization time is one to two and a half hours.

39. The method of claim 37, wherein said enzymatic digestion is for 15 to 30 minutes.

40. The method of claim 37, wherein steps (a) to (c) are performed within less than 200 minutes.

41. The method of claim 37, wherein said at least one MIP comprises a plurality of MIPs corresponding to a plurality of different target regions.

42. The method of claim 37, further comprising at least one of:
(i) sequencing a plurality of synthesized sequences obtained in step (d) and identifying variants of interest; and/or
(ii) applying machine learning algorithm on the identified variants or a subgroup thereof, for calculating sensitivity, specificity and precision thereof, optionally wherein the subgroup of variants comprises variants having VAF below threshold.

43. The method of claim 37, wherein the at least one MIP is a double strand probe.

44. The method of claim 37, wherein said target nucleic acid sequence is at least one of a genomic nucleic acid sequence, a transcriptomic nucleic acid sequence, and a circulating free DNA (cfDNA).

45. The method of claim 37, wherein said target nucleic acid sequence is a nucleic acid sequence associated with, or comprising, at least one of: genetic and/or epigenetic variation/s, pathologic disorder/s, infectious entity, microorganism/s and GC-rich regions, optionally wherein said genetic variations comprise at least one of: single nucleotide variant (SNVs) and/or single-nucleotide polymorphisms (SNPs), insertions and/or deletions, (indels), inversions, copy number variations (CNV), structural variations, alternative splicing, loss of heterozygosity (LOH), gene fusions, translocations, duplications and variable number of tandem repeats.

46. The method of claim 45, wherein at least one of:
(i) said target nucleic acid sequence is associated with at least one hereditary, congenital, and/or somatic pathologic disorder or condition; (ii) said pathologic disorder is at least one of: a neoplastic disorder, a metabolic condition, an inflammatory disorder, an infectious disease caused by a pathogen, mental disorders, an autoimmune disease, a cardiovascular disease, a neurodegenerative disorder, fetal genetic condition and an age-related condition;
(iii) said age related condition is age-related clonal hematopoiesis (ARCH), and wherein said target nucleic acid sequence is a sequence associated with ARCH; and/or
(iv) said at least one target nucleic acid sequence is derived from a genomic DNA of a human subject prone to have ARCH.

47. A method for diagnosing a pathological disorder in a subject by identifying at least one genetic and/or epigenetic variation/s associated with said pathologic disorder, and/or at least one nucleic acid sequence of at least one pathogenic entity, in at least one target nucleic acid sequence of at least one sample of said subject, the method comprising the step of performing molecular inversion probe-based targeted sequencing in at least one test sample of said subject or in any nucleic acid molecule obtained therefrom, wherein the presence of one or more of said variation/s in said target nucleic acid sequence and/or of at least one nucleic acid sequence of at least one pathogenic entity in said sample, indicates that the subject has a risk, is a carrier, or is suffering from said pathologic disorder, and wherein the molecular inversion probe-based targeted sequencing method comprising the step of:
a. contacting at least one MIP with at least one target nucleic acid sequence of said subject, and incubating for a hybridization time of one to three and a half hours, said MIP comprising:
(i) a first region comprising a first sequence complementary to a first target region in said target nucleic acid sequence, and
(ii) a second region comprising a second sequence complementary to a second target region in said target nucleic acid sequence; thereby obtaining a MIP hybridized to the first and second target regions of the target nucleic acid sequence;
b. subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP, wherein the synthesized sequence is further ligated to obtain cyclized product/s in said reaction mixture;
c. subjecting the reaction mixture obtained in step (b) to enzymatic digestion for 10 to 45 minutes, thereby digesting linear MIP/s or nucleic acid molecule/s present in said reaction mixture; and
d. amplifying the synthesized sequence of said cyclized product/s.

48. The method of claim 47, wherein said subject is at least one organism of the biological kingdom Animalia or at least one organism of the biological kingdom Plantae.

49. The method of claim 47, wherein said genetic variations comprise at least one of: SNVs and/or SNP/s, indels, inversions, CNV, LOH, gene fusions, translocations, duplications, structural variations, alternative splicing, variable number of tandem repeats.

50. The method of claim 47, wherein said pathogenic entity is at least one of a viral, a bacterial, a fungal, a parasitic and a protozoan pathogen.

51. The method of claim 47, wherein at least one of:
(i) said target nucleic acid sequence is associated with at least one hereditary, congenital, and/or somatic pathologic disorder or condition; and/or
(ii) said pathologic disorder is at least one of: a neoplastic disorder, a metabolic condition, an inflammatory disorder, an infectious disease caused by a pathogen, an autoimmune-disease, mental disorder, a cardiovascular disease, a neurodegenerative disorder, fetal genetic condition and an age-related condition.

52. A method of detecting the presence of one or more target microorganism, infectious entity in a test sample, the method comprising the step of performing molecular inversion probe-based targeted sequencing according to claim 37, in at least one nucleic acid molecule obtained from said sample, wherein the presence of one or more target nucleic acid sequence associated with said microorganism or infectious entity in said sample indicates the presence thereof in the sample, and wherein the molecular inversion probe-based targeted sequencing method comprising the step of:
a. contacting at least one nucleic acid molecule of the sample with at least one MIP specific for at least one target nucleic acid sequence associated with said microorganism or infectious entity, and incubating for a hybridization time of one to three and a half hours, said MIP comprising:
(iii) a first region comprising a first sequence complementary to a first target region in said target nucleic acid sequence, and
(iv) a second region comprising a second sequence complementary to a second target region in said target nucleic acid sequence; thereby obtaining a MIP hybridized to the first and second target regions of the target nucleic acid sequence;
b. subjecting the hybridized MIP obtained in step (a), to a polymerization reaction in a reaction buffer for 1 to 20 minutes, thereby synthesizing a sequence corresponding to the target nucleic acid sequence nested between the first and second regions of the at least one MIP, wherein the synthesized sequence is further ligated to obtain cyclized product/s in said reaction mixture; and optionally, at least one of:
c. subjecting the reaction mixture obtained in step (b) to enzymatic digestion for 10 to 45 minutes, thereby digesting linear MIP/s or nucleic acid molecule/s present in said reaction mixture; and
d. amplifying the synthesized sequence of said cyclized product/s,

53. The method of claim 52, wherein at least one of:
(i) said microorganism is a prokaryotic microorganism, or a lower eukaryotic microorganism, and wherein said infectious entity is at least one of a viral, a bacterial, a fungal, a parasitic and a protozoan pathogen; and/or
(ii) said sample is a biological sample or an environmental sample.

54. A method of determining the genotype and/or genetic profile of at least one nucleic acid sequence of at least one organism, or at least one infectious entity, the method comprising the step of performing molecular inversion probe-based targeted sequencing according to claim 37 in at least one test sample comprising said at least one nucleic acid sequence, wherein the molecular inversion probe-based targeted sequencing method comprising the step of:
a. contacting at least one MIP with said at least one target nucleic acid sequence, and incubating for a hybridization time of one to three and a half hours, said MIP comprising:
(v) a first region comprising a first sequence complementary to a first target region in said target nucleic acid sequence, and
(vi) a second region comprising a second sequence complementary to a second target region in said target nucleic acid sequence; thereby obtaining a MIP hybridized to the first and second target regions of the target nucleic acid sequence;
b. subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP, wherein the synthesized sequence is further ligated to obtain cyclized product/s in said reaction mixture;
c. subjecting the reaction mixture obtained in step (b) to enzymatic digestion for 10 to 45 minutes, thereby digesting linear MIP/s or nucleic acid molecule/s present in said reaction mixture; and
d. amplifying the synthesized sequence of said cyclized product/s; optionally wherein said organism is at least one organism of at least one of: the biological kingdom Animalia, the biological kingdom Plantae, the biological kingdom Bacteria, the biological kingdom Archaea, the biological kingdom Protozoa, the biological kingdom Chromista and the biological kingdom Fungi.

55. A method for identifying low variant allele frequency (VAF) mutations in a target nucleic acid molecule by performing molecular inversion probe-based targeted sequencing according to claim 37 in said nucleic acid molecule, the method comprising the step of:
a. contacting at least one MIP with at least one target nucleic acid sequence of said nucleic acid molecule, and incubating for a hybridization time of one to three and a half hours, said MIP comprising:
(vii) a first region comprising a first sequence complementary to a first target region in said target nucleic acid sequence, and
(viii) a second region comprising a second sequence complementary to a second target region in said target nucleic acid sequence; thereby obtaining a MIP hybridized to the first and second target regions of the target nucleic acid sequence;
b. subjecting the hybridized MIP obtained in step (a), to a polymerization reaction in a reaction mixture for 1 to 20 minutes, thereby synthesizing a sequence corresponding to the target nucleic acid sequence nested between the first and second regions of the at least one MIP, wherein the synthesized sequence is further ligated to obtain cyclized product/s in the polymerization and/or ligation reaction mixture; and optionally at least one of:
c. subjecting the reaction mixture obtained in step (b) to enzymatic digestion for 10 to 45 minutes, thereby digesting linear MIP/s or nucleic acid molecule/s present in said reaction mixture; and
d. amplifying the synthesized sequence of said cyclized product/s.

56. The method of claim 37, for performing molecular inversion probe-based targeted sequencing in at least one target nucleic acid sequence comprising at least one GC-rich region.
